(19) United States
(12) Patent Application Publication
(10) Pub. No.: US 2025/0326858 A1
(43) Pub. Date: Oct. 23, 2025

(54) 5T4 BINDING AGENTS AND USES THEREOF

(71) Applicant: EXELIXIS, INC., Alameda, CA (US)

(72) Inventors: Bonnie HAMMER, Madison, WI (US); Jeffrey N. HIGAKI, San Mateo, CA (US); Seema KANTAK, Pacifica, CA (US); Charles KAPLAN, San Francisco, CA (US)

(73) Assignee: EXELIXIS, INC., Alameda, CA (US)

(21) Appl. No.: 18/863,565

(22) PCT Filed: May 10, 2023
(86) PCT No.: PCT/US2023/066819

§ 371 Date: Nov. 6, 2024

Related U.S. Application Data
(60) Provisional application No. 63/341,944, filed on May 13, 2022.

(51) Int. Cl. C07K 16/30

(52) U.S. Cl. CPC C07K 16/30; C07K 2317/21; C07K 2317/33; C07K 2317/52; C07K 2317/55; C07K 2317/567; C07K 2317/77; C07K 2317/92; C07K 2317/94; C07K 2319/00

(57) ABSTRACT

The present disclosure provides 5T4 binding agents (e.g., antibodies, including multispecific antibodies such as bispecific antibodies, and antibody-drug conjugates) and uses thereof.

### CROSS-REFERENCE TO RELATED APPLICATIONS

This application claims the benefit of U.S. Provisional Patent Application No. 63/341,944, filed May 13, 2022, the disclosure of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14529-107-228_SEQ_LISTING.xml”, was created on May 8, 2023 and is 102,425 bytes in size.

### FIELD

The present disclosure relates generally to binding agents, such as antibodies or fragments thereof that bind to 5T4 including human 5T4, and methods of use thereof.

### BACKGROUND

5T4 is an N-glycosylated transmembrane 72 kDa glycoprotein containing eight leucine-rich repeats. 5T4, also known as Wnt-Activated Inhibitory Factor 1 or WAIF1, is often referred to as an oncofetal antigen due to its expression in foetal trophoblast (where it was first discovered) or as trophoblast glycoprotein (TPBG). 5T4 is found in tumors including the colorectal, ovarian, and gastric. Its expression is used as a prognostic aid. 5T4 has very limited expression in normal tissue but is widespread in malignant tumours throughout their development. Although its confined expression would appear to give 5T4 the potential to be a target in cancer therapy, therapeutic success with binding agents and vaccines targeting 5T4 has not yet been achieved.

Accordingly, there remains a need in the art for agents that can target 5T4 to treat, prevent, or alleviate 5T4-mediated diseases, disorders, or conditions, including those involving tumor cells expressing 5T4.

### SUMMARY

The present disclosure provides 5T4 binding agents, including human 5T4 binding agents. Such agents include antibodies that bind to 5T4, for example, monospecific or multispecific (e.g., bispecific) antibodies and antibody-drug conjugates (ADCs) that bind to 5T4. Such binding agents, in some embodiments, bind to the same epitope of human 5T4 as an antibody comprising the CDRs described herein (e.g., Tables 1-3). Such binding agents, in some embodiments, bind to the same epitope of human 5T4 as an antibody comprising a heavy chain variable region and a light chain variable region described herein (e.g., Tables 1-3).

The present disclosure also provides compositions comprising a 5T4 binding agent. Such compositions, in some embodiments, include antibodies that bind to 5T4, for example, monospecific or multispecific (e.g., bispecific) antibodies and ADCs that bind to 5T4. Such compositions, in some embodiments, include antibodies and ADCs that bind to essentially the same epitope of human 5T4 as an antibody comprising the CDRs described herein (e.g., Tables 1-3). Such compositions, in some embodiments, include antibodies and ADCs that bind to essentially the same epitope of human 5T4 as an antibody comprising a heavy chain variable region and a light chain variable region described herein (e.g., Tables 1-3).

The present disclosure also provides methods of treating, preventing, or alleviating a 5T4-mediated disease, disorder, or condition, including one or more symptoms of the 5T4-mediated disease, disorder, or condition with a 5T4 binding agent or a composition comprising the agent. Such compositions include antibodies that bind to 5T4, for example, monospecific or multispecific (e.g., bispecific) antibodies and ADCs that bind to 5T4.

### BRIEF DESCRIPTION OF THE DRAWINGS

FIGS. 1A and 1B show a sequence alignment of heavy chain variable regions and light chain variable regions of mAbA4, mAbA15, and mAbA17, including VH CDR1, VH CDR2, VH CDR3, VL CDR1, VL CDR2, and VL CDR3. Boundaries of CDRs are indicated by Kabat, AbM, Chothia, Contact, IMGT and AHon numbering.

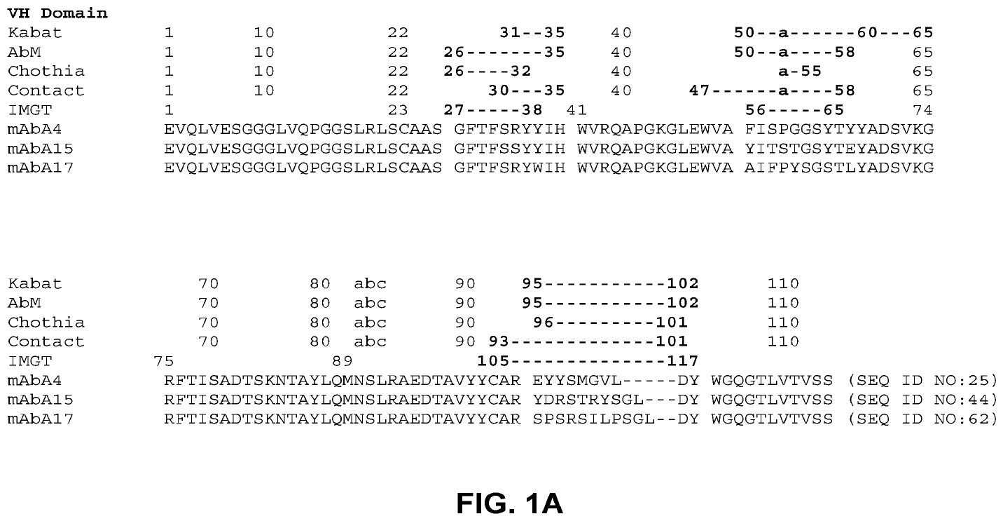

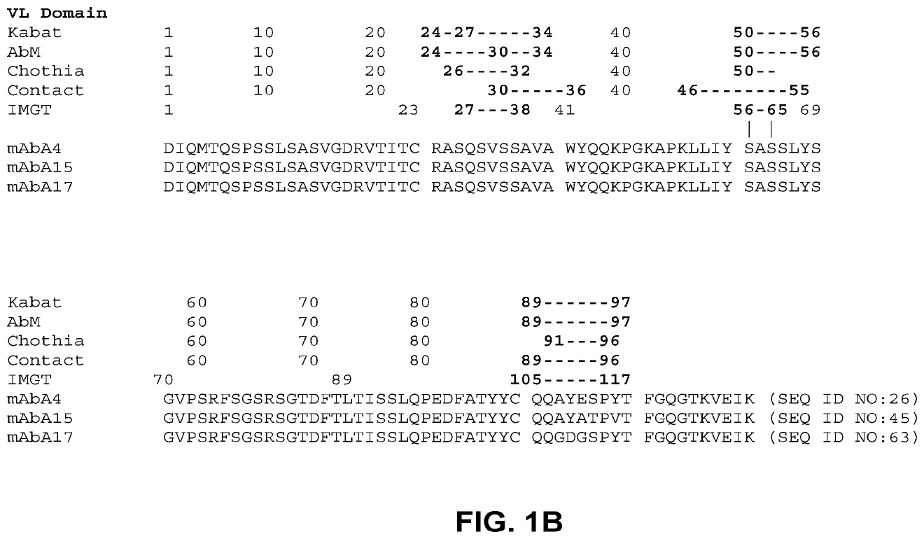

### DETAILED DESCRIPTION

The present disclosure provides 5T4 binding agents. Such agents include antibodies (e.g., monospecific or multispecific, including bispecific) and ADCs that bind to 5T4, including antibodies and ADCs that bind to human 5T4. Such binding agents are useful in compositions and in methods of treating, preventing, or alleviating a 5T4-mediated disease, disorder, or condition, including one or more symptoms of the disease, disorder, or condition. 5T4-mediated diseases, disorders, and conditions include a variety of cancers, including, but not limited to, any cancer wherein the tumor cells express or overexpress 5T4. As used herein, the term “overexpress” means to transcribe and translate more genetic product than normal (such as in normal cells), the process of which is of then a characteristic of cancer cells. In addition, 5T4 binding agents described herein, such as 5T4 binding antibodies (e.g., monospecific or multispecific antibodies, including bispecific antibodies) and 5T4 binding ADCs, are useful for the killing and/or removal of tumor cells. 5T4 binding agents described herein, such as 5T4 binding antibodies (e.g., monospecific or multispecific antibodies, including bispecific antibodies) and 5T4 binding ADCs, are useful in compositions and in methods for treating cancer.

The term “5T4” refers to a polypeptide (“polypeptide” and “protein” are used interchangeably herein) or any native 5T4 from any vertebrate source, including mammals such as primates (e.g., humans, cynomolgus monkey (cyno)), dogs, and rodents (e.g., mice and rats), unless otherwise indicated. 5T4, also known as “5T4 Oncofetal Trophoblast Glycoprotein” or “Wnt-Activated Inhibitory Factor 1” or “5T4 Oncotrophoblast Glycoprotein” or “5T4 Oncofetal Antigen” or “WAIF1” or “M6P1” or “5T4-AG” or “5T4AG” or “TPBG,” is a glycoprotein encoded by the TPBG gene. The term 5T4 encompasses “full-length” 5T4, as well as any form of 5T4 or any fragment thereof that results from processing in the cell. In some embodiments, the 5T4 comprises a signal sequence. In some embodiments, the 5T4 does not include a signal sequence. In some embodiments, the term 5T4 refers to a fragment of the full-length 5T4, which comprises a 5T4 extracellular domain. The term 5T4 also encompasses naturally occurring variants of 5T4, such as SNP variants, splice variants and allelic variants.

The full-length amino acid sequence of human 5T4 is provided below (exemplary signal sequence=italic text; exemplary extracellular domain=underline text):

(SEQ ID NO: 64)
MPGGCSRGPA AGDGRLRLAR LALVLLGWVS SSSPTSSASS FSSSAPFLAS AVSAQPPLPD
QCPALCECSE AARTVKCVNR NLTEVPTDLP AYVRNLFLTG NQLAVLPAGA FARRPPLAEL
AALNLSGSRL DEVRAGAFEH LPSLRQLDLS HNPLADLSPF AFSGSNASVS APSPLVELIL
NHIVPPEDER QNRSFEGMVV AALLAGRALQ GLRRLELASN HFLYLPRDVL AQLPSLRHLD
LSNNSLVSLT YVSFRNLTHL ESLHLEDNAL KVLHNGTLAE LQGLPHIRVF LDNNPWVCDC
HMADMVTWLK ETEVVQGKDR LTCAYPEKMR NRVLLELNSA DLDCDPILPP SLQTSYVFLG
IVLALIGAIF LLVLYLNRKG IKKWMHNIRD ACRDHMEGYH YRYEINADPR LTNLSSNSDV

The full-length amino acid sequence of cynomolgous monkey (cyno) 5T4 is provided below (exemplary signal sequence=italic text; exemplary extracellular domain=underline text):

(SEQ ID NO: 65)
MPGGCSRGPA AGDGRLRLAR LALVLLGWVS SSSSTSSASS SSSSAPFLAS AASAQPPLPD
QCPALCECSE AARTVKCVNR NLTEVPTDLP LYVRNLFLTG NQLAVLPAGA FARRPPLAEL
AALNLSGSRL DEVRGGAFEH LPSLRQLDLS HNPLAYLSPF AFSGSNASIS APSPLVELIL
NHIVPPDDKR QNRSFEGMVA AALVAGRALQ GLHLLELASN HFLYLPRDVL AQLPSLRYLD
LSNNSLVSLT YVSFRNLTHL ESLHLEDNAL KVLHNGTLAE LQGLPHVRVF LDNNPWVCDC
HMADMVTWLK QTGVVQGKDR LTCAFPEKMR NRVLLELNSA DLDCDPILPP SLQTSYVFLG
IVLALIGAIF LLVLYLNRKG IKKWMHNIRD ACRDHMEGYH YRYEINADPR LTNLSSNSDV

The full-length amino acid sequence of mouse 5T4 is provided below (exemplary signal sequence=italic text; exemplary extracellular domain=underline text):

(SEQ ID NO: 66)
MPGAGSRGPS AGDGRLRLAR LALVLLGWVS ASAPSSSVPS SSTSPAAFLA SGSAQPPPAE
RCPAACECSE AARTVKCVNR NLLEVPADLP PYVRNLFLTG NQMTVLPAGA FARQPPLADL
EALNLSGNHL KEVCAGAFEH LPGLRRLDLS HNPLTNLSAF AFAGSNASVS APSPLEELIL
NHIVPPEDQR QNGSFEGMVA FEGMVAAALR SGLALRGLTR LELASNHFLF LPRDLLAQLP
SLRYLDLRNN SLVSLTYASF RNLTHLESLH LEDNALKVLH NSTLAEWHGL AHVKVFLDNN
PWVCDCYMAD MVAWLKETEV VPDKARLTCA FPEKMRNRGL LDLNSSDLDC DAVLPQSLQT
SYVFLGIVLA LIGAIFLLVL YLNRKGIKKW MHNIRDACRD HMEGYHYRYE INADPRLTNL
SSNSDV

As used herein, the term “binding agent” or a grammatical equivalent thereof refers to a molecule (e.g., antibody) with one or more antigen binding sites that binds an antigen. In some embodiments, a 5T4 binding agent as described herein is an antibody, antibody fragment, or other peptide-based molecule as well as a conjugate of an antibody, antibody fragment, or peptide-based molecule (e.g., an ADC) that binds to 5T4, such as human 5T4.

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full-length monoclonal antibodies), antibody compositions with polyepitopic or monoepitopic specificity, recombinantly produced antibodies, single domain (e.g., VHH) antibodies, monospecific antibodies, multispecific antibodies (including bispecific antibodies), synthetic antibodies, chimeric antibodies, humanized antibodies, or human versions of antibodies having full-length heavy and/or light chains. VHH as used herein refers to a domain antibody derived from a variable region of a heavy chain only antibody. Exemplary single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. VHH can also be derived from other species besides Camelidae that may produce heavy chain antibodies naturally devoid of light chain. Antibodies also include antibody fragments (and/or polypeptides that comprise antibody fragments) that retain 5T4 binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable domain antibody, single variable domain, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) variable domains. For example, antibodies also include tetrameric antibodies comprising two heavy chain and two light chain molecules, an antibody light chain monomer, and an antibody heavy chain monomer. Thus, for example, the V region domain may be dimeric and contain VHH-VHH, VH-VH, VH-VL, or VL-VL dimers that bind 5T4. If desired, the VH and VL may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated, for example, into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). In some embodiments, antibodies comprising a VH and/or VL contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monospecific” when used in reference to a binding agent (e.g., an antibody or ADC) as used herein denotes a binding agent that has one or more binding sites each of which binds to the same epitope of the same antigen.

The term “bispecific” when used in reference to a binding agent (e.g., an antibody or ADC) means that the binding agent is able to specifically bind to at least two distinct antigenic determinants, for example two binding sites each formed by a pair of an antibody heavy chain variable domain (VH) and an antibody light chain variable domain (VL) or each formed by a pair of VHH domains binding to different antigens or to different epitopes on the same antigen. Such a bispecific binding agent (e.g., an antibody or ADC) may have a 1+1 format (comprising one binding site for a first antigen or epitope and one binding site for a second antigen or epitope). Other bispecific binding agent (e.g., an antibody or ADC) formats may be 2+1 or 1+2 formats (comprising two binding sites for a first antigen or epitope and one binding site for a second antigen or epitope) or 2+2 format (comprising two binding sites for a first antigen or epitope and two binding sites for a second antigen or epitope). When a bispecific binding agent (e.g., an antibody or ADC) comprises two antigen binding sites, each may bind to a different antigenic determinant. Such a bispecific binding agent (e.g., an antibody or ADC) may bind to two different epitopes on the same antigen (e.g., epitopes on 5T4).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, or at least 90%, and in some embodiments at least 95%, 96%, 97%, 98%, or 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full-length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component or a drug (e.g., toxin). Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains or dimers of single chains.

As used herein, an “antigen” is a moiety or molecule that contains an epitope to which a binding agent (e.g., an antibody or ADC) can bind. As such, an antigen can be bound by an antibody. In some embodiments, the antigen, to which a binding agent (e.g., an antibody or ADC) described herein binds, is 5T4 (e.g., human 5T4), or a fragment thereof, including a fragment that comprises one or more domains of 5T4.

As used herein, an “epitope” is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human 5T4. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three-dimensional protein structure. In other embodiments, an antibody requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities. Additional details regarding methods for epitope binning and determining epitope binding of antibodies are described herein, as shown in Example 5.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, BIACORE™, KinExA 3000 instrument (Sapidyne Instruments, Boise, ID), the OctetQK384 system (ForteBio, Menlo Park, CA), or other assays known in the art. In some embodiments, an antibody or antigen binding domain binds to or specifically binds to an antigen when it binds to the antigen with higher affinity than to any cross-reactive antigen as determined using experimental techniques, such as radioimmunoassays (RIAs) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen binding domain to a “non-target”protein is less than about 10% of the binding of the antibody or antigen binding domain to its particular target antigen, for example, as determined by fluorescence activated cell sorting (FACS) analysis or RIAs. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In another specific embodiment, molecules that specifically bind to an antigen do not cross react with other non-5T4 proteins. In some embodiments “specifically binds” means, for instance, that a polypeptide or molecule binds a protein or target with a K_{D} of about 0.1 mM or less, but more usually less than about 1 μM. In some embodiments, “specifically binds” means that a polypeptide or molecule binds a target with a K_{D} of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding agent such as an antibody or ADC) and its binding partner (e.g., an antigen such as 5T4). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by biolayer interferometry (BLI) using, for example, the OctetQK384 system (ForteBio, Menlo Park, CA). Alternatively, the K_{D} may also be measured in a radiolabeled antigen binding assay (RIA), for example, performed with the Fab version of an antibody of interest and its antigen (Chen, et al., (1999) J. Mol Biol 293:865-881) or using surface plasmon resonance (SPR) assays by BIACORE™ using, for example, a BIACORE™-2000 or a BIACORE™-3000 BIACORE™, Inc., Piscataway, NJ). An “on-rate” or “rate of association” or “association rate” or “kₒₙ,” as well as an “off-rate” or “rate of dissociation” or “dissociation rate” or “k_{off},” can also be determined with the same SPR or BLI techniques described above using, for example, the OctetQK384 system (ForteBio, Menlo Park, CA) or a BIACORE™-2000 or a BIACORE™-3000 (BIACORE™, Inc., Piscataway, NJ), respectively.

The term “compete” when used in the context of 5T4 binding agents (e.g., antibodies or ADCs) means binding agents that compete for the same epitope or binding site on a target, which includes competition between such binding agents as determined by an assay in which the binding agent under study prevents or inhibits the specific binding of a reference molecule (e.g., a reference ligand, or reference antigen binding protein, such as a reference antibody) to a common antigen (e.g., 5T4). Numerous types of competitive binding assays can be used to determine if a test binding agent competes with a reference molecule for binding to 5T4 (e.g., human 5T4). Examples of assays that can be employed include solid phase direct or indirect radioimmunoassay (RIA); solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619 or Cheung, et al., (1990) Virology 176:546-552); solid phase direct labeled assay; solid phase direct labeled sandwich assay (see, e.g., Harlow and Lane, (1988) Antibodies, A Laboratory Manual, Cold Spring Harbor Press); solid phase direct label RIA using I-125 label (see, e.g., Morel et al., (1988) Molec. Immunol. 25:7-15); and direct labeled RIA (Moldenhauer et al., (1990) Scand. J. Immunol. 32:77-82). Typically, such an assay involves the use of a purified antigen (e.g., 5T4, such as human 5T4) bound to a solid surface or cells bearing either of an unlabelled test antigen binding protein (e.g., test 5T4 antibody or ADC) or a labeled reference antigen binding protein (e.g., reference 5T4 antibody or ADC). Competitive inhibition may be measured by determining the amount of label bound to the solid surface or cells in the presence of the test antigen binding protein. Usually, the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70% or 75%. In some instance, binding is inhibited by at least 80%, 85%, 90%, 95%, 96%, 97%, 98%, 99% or more.

As used herein, the term “constant region” or “constant domain” is a well-known antibody term of art and refers to an antibody portion, for example, a carboxyl terminal portion of a light and/or heavy chain which is not directly involved in binding of an antibody to an antigen but which can exhibit various effector functions, such as interaction with an Fc receptor. The term includes the portion of an immunoglobulin molecule having a generally more conserved amino acid sequence relative to an immunoglobulin variable domain.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. An exemplary Fc region sequence is provided below (CH2 domain=bold text; CH3 domain=underline text):

(SEQ ID NO: 67)
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, (e.g., substituting, addition, or deletion) preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region described herein can possess at least about 80% homology with a native sequence Fc region and/or with an Fc region of a parent polypeptide, or at least about 90% homology therewith, for example, at least about 95% homology therewith. The variant Fc region herein described herein may have a loss of effector function (e.g., silent Fc). An exemplary variant Fc region (“silent Fc”) sequence is provided below (CH2 domain=bold text with amino acid changes underlined; CH3 domain=underline text):

(SEQ ID NO: 68)
CPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALKAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK.

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) or lambda (A) based on the amino acid sequence of the constant domains. Light chain amino acid sequences are well known in the art.

The terms “antigen binding fragment,” “antigen binding domain,” “antigen binding region,” and similar terms refer to that portion of an antibody, which comprises the amino acid residues that interact with an antigen and confer on the binding fragment, domain, or region its specificity and affinity for the antigen (e.g., the CDRs). “Antigen binding fragment” as used herein includes “antibody fragment,” which comprises a portion of an antibody including one or more CDRs, such as the antigen binding or variable region of the antibody.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen binding sites that bind to a 5T4 antigen.

Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or a subclass thereof.

In some embodiments, an antibody is a 4-chain antibody unit comprising two heavy (H) chain/light (L) chain pairs, wherein the amino acid sequences of the H chains are identical and the amino acid sequences of the L chains are identical. In some embodiments, an antibody is a 2-chain antibody unit comprising a VHH-VHH pair, wherein the amino acid sequences of the VHH are identical. In some embodiments, the H and/or L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprise a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

An antibody or fragment thereof may preferentially bind to 5T4, such as human 5T4, meaning that the antibody or fragment thereof binds 5T4 with greater affinity than it binds to an unrelated control protein and/or binds human 5T4 with greater affinity than it binds to an unrelated control protein. For example, the antibody or fragment thereof may specifically recognize and bind 5T4 or a portion thereof. “Specific binding” means that the antibody or fragment thereof binds to 5T4 with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind 5T4 substantially exclusively (e.g., is able to distinguish 5T4 from other known polypeptides, for example, by virtue of measurable differences in binding affinity). In some embodiments, a 5T4 binding agent (e.g., an antibody or ADC) may react with 5T4 sequences other than human 5T4 sequences (e.g., cynomolgous 5T4 sequences).

The term “variable region” or “variable domain” refers to a portion of the light or heavy chains of an antibody that is generally located at the amino-terminal of the light or heavy chain, has a length of about 120 to 130 amino acids in the heavy chain and about 100 to 110 amino acids in the light chain, and is used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions.” The variable regions of heavy and light chains each comprise four frameworks (FR1, FR2, FR3 and FR4), largely adopting a p sheet configuration, connected by three hypervariable regions, which form loops connecting, and in some cases forming part of, the p sheet structure. The hypervariable regions in each chain are held together in close proximity by the frameworks and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, (1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “hypervariable region,” “HVR,” “HV,” “complementarity determining region,” or “CDR” when used herein refers to the regions of an antibody variable region that are hypervariable in sequence and/or form structurally defined loops. Generally, antibodies comprise six hypervariable regions: three in the VH (H1 or VH CDR1, H2 or VH CDR2, H3 or VH CDR3), and three in the VL (L1 or VL CDR1, L2 or VL CDR2, L3 or VL CDR3). A number of hypervariable region delineations are in use and are encompassed herein. The Kabat CDRs are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT®) Information System (Lefranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra) and is also illustrated below. Various systems known in the art or described herein represent different ways of delineating CDRs, and when they are used to define the same antibody, they are often considered equivalent. An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

The term “vector” refers to a substance that is used to carry or include a nucleic acid sequence, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and/or inducible promoters, transcription enhancers, transcription terminators, and the like which are well known in the art. When two or more nucleic acid molecules are to be co-expressed (e.g. both an antibody heavy and light chain or an antibody VH and VL) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a 5T4 binding agent as described herein), and it is further understood that expression levels can be optimized to obtain sufficient expression using methods well known in the art.

A “5T4-mediated disease,” “5T4-mediated disorder” and “5T4-mediated condition” are used interchangeably and refer to any disease, disorder or condition associated with or characterized by 5T4-expressing cells, such as 5T4-expressing tumor cells. A 5T4-mediated disease includes a cancer including but not limited to cancers that express or overexpress 5T4.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., an antibody or ADC described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the prophylactic or therapeutic effect of another therapy (e.g., a therapy other than the administration of an antibody or ADC described herein). A “therapeutically effective amount” of a substance/molecule/agent of the present disclosure (e.g., a 5T4 antibody or ADC) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an antibody or other agent (e.g., ADC or drug) effective to “treat” a disease, disorder, or condition, in a subject or mammal.

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s).

The full therapeutic or prophylactic effect does not necessarily occur by administration of one dose, and may occur only after administration of a series of doses. Thus, a therapeutically or prophylactically effective amount may be administered in one or more administrations.

The term “pharmaceutically acceptable” as used herein means being approved by a regulatory agency of the federal or a state government, or listed in the U.S. Pharmacopeia, European Pharmacopeia or other generally recognized Pharmacopeia for use in animals, and more particularly in humans.

“Carriers” as used herein include carriers, excipients, stabilizers, or preservatives that are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington's Pharmaceutical Sciences (1990) Mack Publishing Co., Easton, PA. Compositions, including pharmaceutical compounds, may contain a prophylactically or therapeutically effective amount of a 5T4 binding agent (e.g., an antibody or ADC), for example, in isolated or purified form, together with a suitable amount of carrier so as to provide the form for proper administration to the subject (e.g., patient). The formulation should suit the mode of administration.

In some embodiments, the present disclosure provides 5T4 binding agents that can be used herein as therapeutic agents. Such agents include antibodies (e.g., monospecific or multispecific, including bispecific) and ADCs that bind to 5T4. Exemplary antibodies include polyclonal, monoclonal, humanized, human, bispecific, and heteroconjugate antibodies, as well as variants thereof having increased or decreased affinity or other properties.

In some embodiments, described herein are 5T4 binding agents (e.g., antibodies or ADCs) that bind to 5T4, including a 5T4 polypeptide, a 5T4 polypeptide fragment, a 5T4 peptide or a 5T4 epitope. In some embodiments, the 5T4 binding agents are human or humanized antibodies (e.g., comprising human constant regions) that bind 5T4, including a 5T4 polypeptide, a 5T4 polypeptide fragment, a 5T4 peptide or a 5T4 epitope. In some embodiments, a 5T4 binding agent (e.g., an antibody or ADC), such as a human 5T4 binding agent, can bind to 5T4 expressed on the surface of a mammalian (e.g., human) cell, including a 5T4 expressing tumor cell. In some embodiments, a 5T4 binding agent (e.g., an antibody or ADC) binds a 5T4 extracellular epitope exposed on a cell such as a tumor cell (e.g., a 5T4 epitope). In some embodiments, described herein is a 5T4 binding agent (e.g., an antibody or ADC) that binds to 5T4, such as human 5T4 or portions thereof. In some embodiments, 5T4 is a human 5T4. In some embodiments, a 5T4 binding agent is a human 5T4 binding agent (e.g., an antibody or ADC that binds to human 5T4). An exemplary amino acid sequence of human 5T4 is described herein.

In some embodiments, the 5T4 binding agents (e.g., antibodies or ADCs) described herein compete for the binding to 5T4, such as human 5T4, with a 5T4 binding agent (e.g., an antibody or ADC) that comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in Tables 1-3. Accordingly, in some embodiments, a 5T4 binding agent (e.g., an antibody or ADC) described herein competes for the binding to 5T4, such as human 5T4, with a 5T4 binding agent (e.g., an antibody or ADC) that comprises one, two, and/or three VH CDRs and/or one, two, and/or three VL CDRs from: (a) the antibody designated mAbA4; (b) the antibody designated mAbA15; and (c) the antibody designated mAbA17 as shown in Tables 1-3. In some embodiments, a 5T4 binding agent (e.g., an antibody or ADC) described herein competes for the binding to 5T4, such as human 5T4, with a 5T4 binding agent (e.g., an antibody or ADC) that comprises one, two, and/or three VH CDRs and one, two, and/or three VL CDRs from: (a) the antibody designated mAbA4; (b) the antibody designated mAbA15; and (c) the antibody designated mAbA17; as shown in Tables 1-3. In some embodiments, a 5T4 binding agent (e.g., an antibody or ADC) described herein competes for the binding to 5T4, such as human 5T4, with a 5T4 binding agent (e.g., an antibody or ADC) that comprises a VH region and VL region from: (a) the antibody designated mAbA4; (b) the antibody designated mAbA15; and (c) the antibody designated mAbA17 as shown in Tables 1-3. In some embodiments, a 5T4 binding agent (e.g., an antibody or ADC) described herein competes for the binding to 5T4, such as human 5T4, with a 5T4 binding agent (e.g., an antibody or ADC) that comprises: (a) a VH region comprising the amino acid sequence of SEQ ID NO:25 and a VL region comprising the amino acid sequence of SEQ ID NO:26; (b) a VH region comprising the amino acid sequence of SEQ ID NO:44 and a VL region comprising the amino acid sequence of SEQ ID NO:45; and (c) a VH region comprising the amino acid sequence of SEQ ID NO:62 and a VL region comprising the amino acid sequence of SEQ ID NO:63.

In some embodiments, the 5T4 binding agents (e.g., antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in Tables 1-3. Accordingly, in some embodiments, a 5T4 binding agent (e.g., an antibody or ADC) described herein comprises one, two, and/or three heavy chain CDRs and/or one, two, and/or three light chain CDRs from: (a) the antibody designated mAbA4; (b) the antibody designated mAbA15; and (c) the antibody designated mAbA17 as shown in Tables 1-3. In some embodiments, a 5T4 binding agent (e.g., an antibody or ADC) described herein comprises one, two, and/or three heavy chain CDRs and one, two, and/or three light chain CDRs from: (a) the antibody designated mAbA4; (b) the antibody designated mAbA15; and (c) the antibody designated mAbA17 as shown in Tables 1-3.

In some embodiments, a 5T4 binding agent (e.g., an antibody or ADC) comprises a VH region, which comprises VH CDR1, VH CDR2, and/or VH CDR3, and/or a VL region, which comprises VL CDR1, VL CDR2, and/or VL CDR3, of any one of the binding agents described herein (see, e.g., any one of Tables 1-3). Accordingly, in some embodiments, a 5T4 binding agent (e.g., an antibody or ADC) described herein comprises one, two, and/or three heavy chain CDRs and/or one, two, and/or three light chain CDRs from Table 1. In some embodiments, a 5T4 binding agent (e.g., an antibody or ADC) described herein comprises one, two, and/or three heavy chain CDRs and/or one, two, and/or three light chain CDRs from Table 2. In some embodiments, a 5T4 binding agent (e.g., an antibody or ADC) described herein comprises one, two, and/or three heavy chain CDRs and/or one, two, and/or three light chain CDRs from Table 3.

The antibody designated mAbA4 comprises a VH sequence that is SEQ ID NO:25 and a VL sequence that is SEQ ID NO:26.

The antibody designated mAbA15 comprises a VH sequence that is SEQ ID NO:44 and a VL sequence that is SEQ ID NO:45.

The antibody designated mAbA17 comprises a VH sequence that is SEQ ID NO:62 and a VL sequence that is SEQ ID NO:63.

TABLE 1
Antibody mAbA4

| | | Exemplary | IMGT | Kabat | Chothia | Contact | AbM |
|---|---|---|---|---|---|---|---|
| VH CDR | VH | GFTFSRYYIH | GFTFSRYY | RYYIH | GFTFSRY | SRYYIH | GFTFSRYYIH |
| Seq. | CDR1 | (SEQ ID NO: 1) | (SEQ ID NO: 7) | (SEQ ID NO: 12) | (SEQ ID NO: 13) | (SEQ ID NO: 18) | (SEQ ID NO: 1) |
| | VH | FISPGGSYTYYAD | ISPGGSYT | FISPGGSYTYYAD | PGGS | WVAFISPGGSYT | FISPGGSYTY |
| | CDR2 | SVKG | (SEQ ID NO: 8) | SVKG | (SEQ ID NO: 14) | Y | (SEQ ID NO: 24) |
| | | (SEQ ID NO: 2) | | (SEQ ID NO: 2) | | (SEQ ID NO: 19) | |
| | VH | EYYSMGVLDY | AREYYSMGVLD | EYYSMGVLDY | YYSMGVLD | AREYYSMGVLD | EYYSMGVLDY |
| | CDR3 | (SEQ ID NO: 3) | Y | (SEQ ID NO: 3) | (SEQ ID NO: 15) | (SEQ ID NO: 20) | (SEQ ID NO: 3) |
| | | | (SEQ ID NO: 9) | | | | |
| VL CDR | VL | RASQSVSSAVA | QSVSSA | RASQSVSSAVA | SQSVSSA | SSAVAWY | RASQSVSSAVA |
| Seq. | CDR1 | (SEQ ID NO: 4) | (SEQ ID NO: 10) | (SEQ ID NO: 4) | (SEQ ID NO: 16) | (SEQ ID NO: 21) | (SEQ ID NO: 4) |
| | VL | SASSLYS | SAS | SASSLYS | SAS | LLIYSASSLY | SASSLYS |
| | CDR2 | (SEQ ID NO: 5) | (SEQ ID NO: 11) | (SEQ ID NO: 5) | (SEQ ID NO: 11) | (SEQ ID NO: 22) | (SEQ ID NO: 5) |
| | VL | QQAYESPYT | QQAYESPYT | QQAYESPYT | AYESPY | QQAYESPY | QQAYESPYT |
| | CDR3 | (SEQ ID NO: 6) | (SEQ ID NO: 6) | (SEQ ID NO: 6) | (SEQ ID NO: 17) | (SEQ ID NO: 23) | (SEQ ID NO: 6) |

VH Sequence:
EVQLVESGGGLVQPGGSLRLSCAASGFTFSRYYIHWVRQAPGKGLEWVAFISPGGSYTYYADSVKGRFTISADTSKNTAYLQMNSLRAEDTAVYY
CAREYYSMGVLDYWGQGTLVTVSS (SEQ ID NO: 25)
VL Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQAYESPY
TFGQGTKVEIK (SEQ ID NO: 26)

TABLE 2
Antibody mAbA15

| | | Exemplary | IMGT | Kabat | Chothia | Contact | AbM |
|---|---|---|---|---|---|---|---|
| VH CDR | VH | GFTFSSYYIH | GFTFSSYY | SYYIH | GFTFSSY | SSYYIH | GFTFSSYYIH |
| Seq. | CDR1 | (SEQ ID NO: 27) | (SEQ ID NO: 31) | (SEQ ID NO: 34) | (SEQ ID NO: 35) | (SEQ ID NO: 39) | (SEQ ID NO: 27) |
| | VH | YITSTGSYTEYADS | ITSTGSYT | YITSTGSYTEYADS | STGS | WVAYITSTGSYT | YITSTGSYTE |
| | CDR2 | VKG | (SEQ ID NO: 32) | VKG | (SEQ ID NO: 36) | E | (SEQ ID NO: 43) |
| | | (SEQ ID NO: 28) | | (SEQ ID NO: 28) | | (SEQ ID NO: 40) | |
| | VH | YDRSTRYSGLDY | ARYDRSTRYSG | YDRSTRYSGLDY | DRSTRYSGLD | ARYDRSTRYSGL | YDRSTRYSGLD |
| | CDR3 | (SEQ ID NO: 29) | LDY | (SEQ ID NO: 29) | (SEQ ID NO: 37) | D | Y |
| | | | (SEQ ID NO: 33) | | | (SEQ ID NO: 41) | (SEQ ID NO: 29) |
| VL CDR | VL | RASQSVSSAVA | QSVSSA | RASQSVSSAVA | SQSVSSA | SSAVAWY | RASQSVSSAVA |
| Seq. | CDR1 | (SEQ ID NO: 4) | (SEQ ID NO: 10) | (SEQ ID NO: 4) | (SEQ ID NO: 16) | (SEQ ID NO: 21) | (SEQ ID NO: 4) |
| | VL | SASSLYS | SAS | SASSLYS | SAS | LLIYSASSLY | SASSLYS |
| | CDR2 | (SEQ ID NO: 5) | (SEQ ID NO: 11) | (SEQ ID NO: 5) | (SEQ ID NO: 11) | (SEQ ID NO: 22) | (SEQ ID NO: 5) |
| | VL | QQAYATPVT | QQAYATPVT | QQAYATPVT | AYATPV | QQAYATPV | QQAYATPVT |
| | CDR3 | (SEQ ID NO: 30) | (SEQ ID NO: 30) | (SEQ ID NO: 30) | (SEQ ID NO: 38) | (SEQ ID NO: 42) | (SEQ ID NO: 30) |

VH Sequence:
EVQLVESGGGLVQPGGSLRLSCAASGFTFSSYYIHWVRQAPGKGLEWVAYITSTGSYTEYADSVKGRFTISADTSKNTAYLQMNSLRAEDTAVYY
CARYDRSTRYSGLDYWGQGTLVTVSS (SEQ ID NO: 44)
VL Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQAYATPV
TFGQGTKVEIK (SEQ ID NO: 45)

TABLE 3
Antibody mAbA17

| | | Exemplary | IMGT | Kabat | Chothia | Contact | AbM |
|---|---|---|---|---|---|---|---|
| VH CDR | VH | GFTFSRYWIH | GFTFSRYW | RYWIH | GFTFSRY | SRYWIH | GFTFSRYWIH |
| Seq. | CDR1 | (SEQ ID NO: 46) | (SEQ ID NO: 50) | (SEQ ID NO: 53) | (SEQ ID NO: 13) | (SEQ ID NO: 57) | (SEQ ID NO: 46) |
| | VH | AIFPYSGSTLYADS | IFPYSGST | AIFPYSGSTLYAD | PYSG | WVAAIFPYSGST | AIFPYSGSTL |
| | CDR2 | VKG | (SEQ ID NO: 51) | SVKG | (SEQ ID NO: 54) | L | (SEQ ID NO: 61) |
| | | (SEQ ID NO: 47) | | (SEQ ID NO: 47) | | (SEQ ID NO: 58) | |
| | VH | SPSRSILPSGLDY | ARSPSRSILPSGL | SPSRSILPSGLDY | PSRSILPSGLD | ARSPSRSILPSG | SPSRSILPSGLDY |
| | CDR3 | (SEQ ID NO: 48) | DY | (SEQ ID NO: 48) | (SEQ ID NO: 55) | LD | (SEQ ID NO: 48) |
| | | | (SEQ ID NO: 52) | | | (SEQ ID NO: 59) | |
| VL CDR | VL | RASQSVSSAVA | QSVSSA | RASQSVSSAVA | SQSVSSA | SSAVAWY | RASQSVSSAVA |
| Seq. | CDR1 | (SEQ ID NO: 4) | (SEQ ID NO: 10) | (SEQ ID NO: 4) | (SEQ ID NO: 16) | (SEQ ID NO: 21) | (SEQ ID NO: 4) |
| | VL | SASSLYS | SAS | SASSLYS | SAS | LLIYSASSLY | SASSLYS |
| | CDR2 | (SEQ ID NO: 5) | (SEQ ID NO: 11) | (SEQ ID NO: 5) | (SEQ ID NO: 11) | (SEQ ID NO: 22) | (SEQ ID NO: 5) |
| | VL | QQGDGSPYT | QQGDGSPYT | QQGDGSPYT | GDGSPY | QQGDGSPY | QQGDGSPYT |
| | CDR3 | (SEQ ID NO: 49) | (SEQ ID NO: 49) | (SEQ ID NO: 49) | (SEQ ID NO: 56) | (SEQ ID NO: 60) | (SEQ ID NO: 49) |

VH Sequence:
EVQLVESGGGLVQPGGSLRLSCAASGFTFSRYWIHWVRQAPGKGLEWVAAIFPYSGSTLYADSVKGRFTISADTSKNTAYLQMNSLRAEDTAVYY
CARSPSRSILPSGLDYWGQGTLVTVSS (SEQ ID NO: 62)
VL Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQGDGSPY
TFGQGTKVEIK (SEQ ID NO: 63)

In some embodiments, 5T4 binding agents (e.g., antibodies such as bispecific antibodies or ADCs), including human 5T4 binding agents, described herein comprise a VH region or VH domain. Additionally or alternatively, in some embodiments, 5T4 binding agents (e.g., antibodies such as bispecific antibodies or ADCs), including human 5T4 binding agents, described herein comprise a VL region or VL domain. In some embodiments, 5T4 binding agents (e.g., antibodies such as bispecific antibodies or ADCs), including human 5T4 binding agents, described herein have a combination of (i) a VH domain or VH region; and/or (ii) a VL domain or VL region.

In some embodiments, 5T4 binding agents (e.g., antibodies such as bispecific antibodies or ADCs), including human 5T4 binding agents, described herein comprise a heavy chain having a combination of (i) a VH described in any one of Tables 1-3; and (ii) one or more heavy chain constant domains (e.g., CH1, Hinge, CH2, and CH3). An exemplary IgG heavy chain comprises any VH sequence as described herein and the following CH1, Hinge, CH2, and CH3 amino acid sequence:

(SEQ ID NO: 69)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

Another exemplary IgG heavy chain comprises any VH sequence as described herein and the following CH1, Hinge, CH2, and CH3 amino acid sequence:

(SEQ ID NO: 70)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALKAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, 5T4 binding agents (e.g., antibodies such as bispecific antibodies or ADCs), including human 5T4 binding agents, described herein comprise a light chain having a combination of (i) a VL domain described in any one of Tables 1-3; and (ii) a light chain constant domain (CL). An exemplary light chain (e.g., for pairing with an IgG heavy chain) comprises any VL sequence described herein and the following CL amino acid sequence:

(SEQ ID NO: 71)
RTVAAPSVFIFPPSDSQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some embodiments, 5T4 binding agents (e.g., antibodies such as bispecific antibodies or ADCs), including human 5T4 binding agents, described herein comprise (a) a heavy chain having a combination of (i) a VH described in any one of Tables 1-3, and (ii) one or more heavy chain constant domains (e.g., CH1, Hinge, CH2, and CH3); and (b) a light chain having a combination of (i) a VL described in any one of Tables 1-3, and (ii) a light chain constant domain in an IgG format (CL or CL1). An exemplary 5T4 binding agent (e.g., an antibody or ADC) comprises an IgG heavy chain comprising any VH sequence as described herein and the amino acid sequence of SEQ ID NO:69 or 70, and a light chain comprising any VL sequence as described herein and the amino acid sequence of SEQ ID NO:71.

In some embodiments, 5T4 binding agents (e.g., antibodies such as bispecific antibodies or ADCs), including a human 5T4 binding agent, described herein comprises one or more CDRs, including six CDRs, for example, VH CDR1, VH CDR2, VH CDR3, VL CDR1, VL CDR2, and/or VL CDR3 identified in Table 1. In some embodiments, 5T4 binding agents (e.g., antibodies such as bispecific antibodies or ADCs), including a human 5T4 binding agent, described herein comprises one or more CDRs, including six CDRs, for example, VH CDR1, VH CDR2, VH CDR3, VL CDR1, VL CDR2, and/or VL CDR3 identified in Table 2. In some embodiments, 5T4 binding agents (e.g., antibodies such as bispecific antibodies or ADCs), including a human 5T4 binding agent, described herein comprises one or more CDRs, including six CDRs, for example, VH CDR1, VH CDR2, VH CDR3, VL CDR1, VL CDR2, and/or VL CDR3 identified in Table 3. In some embodiments, 5T4 binding agents (e.g., antibodies such as bispecific antibodies or ADCs), including a human 5T4 binding agent, described herein comprises one or more CDRs, including six CDRs, for example, VH CDR1, VH CDR2, VH CDR3, VL CDR1, VL CDR2, and/or VL CDR3 identified in Tables 1, 2, and/or 3.

In some embodiments, 5T4 binding agents (e.g., antibodies such as bispecific antibodies or ADCs), including human 5T4 binding agents, described herein comprise one or more CDRs, including three VH CDRs, for example, VH CDR1, VH CDR2, VH CDR3, listed in Table 1. In other embodiments, 5T4 binding agents (e.g., antibodies such as bispecific antibodies or ADCs), including human 5T4 binding agents, described herein comprise one or more CDRs, including three VL CDRs, for example, VL CDR1, VL CDR2, and/or VL CDR3, listed in Table 1. In yet other embodiments, 5T4 binding agents (e.g., antibodies such as bispecific antibodies or ADCs), including human 5T4 binding agents, described herein comprise one or more CDRs, including three VH CDRs, for example, VH CDR1, VH CDR2, VH CDR3, listed in Table 1 and one or more CDRs, including three VL CDRs, for example, VL CDR1, VL CDR2, and/or VL CDR3, listed in Table 1.

In some embodiments, 5T4 binding agents (e.g., antibodies such as bispecific antibodies or ADCs), including human 5T4 binding agents, described herein comprise one or more CDRs, including three VH CDRs, for example, VH CDR1, VH CDR2, VH CDR3, listed in Table 2. In other embodiments, 5T4 binding agents (e.g., antibodies such as bispecific antibodies or ADCs), including human 5T4 binding agents, described herein comprise one or more CDRs, including three VL CDRs, for example, VL CDR1, VL CDR2, and/or VL CDR3, listed in Table 2. In yet other embodiments, 5T4 binding agents (e.g., antibodies such as bispecific antibodies or ADCs), including human 5T4 binding agents, described herein comprise one or more CDRs, including three VH CDRs, for example, VH CDR1, VH CDR2, VH CDR3, listed in Table 2 and one or more CDRs, including three VL CDRs, for example, VL CDR1, VL CDR2, and/or VL CDR3, listed in Table 2.

In some embodiments, 5T4 binding agents (e.g., antibodies such as bispecific antibodies or ADCs), including human 5T4 binding agents, described herein comprise one or more CDRs, including three VH CDRs, for example, VH CDR1, VH CDR2, VH CDR3, listed in Table 3. In other embodiments, 5T4 binding agents (e.g., antibodies such as bispecific antibodies or ADCs), including human 5T4 binding agents, described herein comprise one or more CDRs, including three VL CDRs, for example, VL CDR1, VL CDR2, and/or VL CDR3, listed in Table 3. In yet other embodiments, 5T4 binding agents (e.g., antibodies such as bispecific antibodies or ADCs), including human 5T4 binding agents, described herein comprise one or more CDRs, including three VH CDRs, for example, VH CDR1, VH CDR2, VH CDR3, listed in Table 3 and one or more CDRs, including three VL CDRs, for example, VL CDR1, VL CDR2, and/or VL CDR3, listed in Table 3.

In some embodiments, a 5T4 binding agent (e.g., an antibody, such as a bispecific antibody) described herein comprises one or more complementarity determining regions (CDRs) comprising an amino acid sequence selected from a group consisting of SEQ ID NOS: 1-24, 27-43, or 46-61. In some embodiments, a 5T4 binding agent (e.g., an antibody such as a bispecific antibody or ADC) described herein comprises two or more complementarity determining regions (CDRs) comprising an amino acid sequence selected from a group consisting of SEQ ID NOS: 1-24, 27-43, or 46-61. In some embodiments, a 5T4 binding agent (e.g., an antibody such as a bispecific antibody or ADC) described herein comprises three or more complementarity determining regions (CDRs) comprising an amino acid sequence selected from a group consisting of SEQ ID NOS: 1-24, 27-43, or 46-61. In some embodiments, a 5T4 binding agent (e.g., an antibody such as a bispecific antibody or ADC) described herein comprises four or more complementarity determining regions (CDRs) comprising an amino acid sequence selected from a group consisting of SEQ ID NOS: 1-24, 27-43, or 46-61. In some embodiments, a 5T4 binding agent (e.g., an antibody such as a bispecific antibody or ADC) described herein comprises five or more complementarity determining regions (CDRs) comprising an amino acid sequence selected from a group consisting of SEQ ID NOS: 1-24, 27-43, or 46-61. In some embodiments, a 5T4 binding agent (e.g., an antibody such as a bispecific antibody or ADC) described herein comprises six or more complementarity determining regions (CDRs) comprising an amino acid sequence selected from a group consisting of SEQ ID NOS: 1-24, 27-43, or 46-61.

In some embodiments, a 5T4 binding agent (e.g., an antibody such as a bispecific antibody or ADC) described herein comprise one or more (e.g., one, two or three) VH CDRs listed in Tables 1-3. In other embodiments, a 5T4 binding agent (e.g., an antibody such as a bispecific antibody or ADC) described herein comprises one or more (e.g., one, two or three) VL CDRs listed in Tables 1-3. In yet other embodiments, a 5T4 binding agent (e.g., an antibody such as a bispecific antibody or ADC) described herein comprises one or more (e.g., one, two or three) VH CDRs listed in Tables 1-3 and one or more VL CDRs listed in Tables 1-3. Accordingly, in some embodiments, a 5T4 binding agent (e.g., an antibody such as a bispecific antibody or ADC) described herein comprises a VH CDR1 having the amino acid sequence of any one of SEQ ID NOS:1, 7, 12, 13, 18, 27, 31, 34, 35, 39, 46, 50, 53, and 57. In some embodiments, a 5T4 binding agent (e.g., an antibody such as a bispecific antibody or ADC) described herein comprises a VH CDR2 having the amino acid sequence of any one of SEQ ID NOS:2, 8, 14, 19, 24, 28, 32, 36, 40, 43, 47, 51, 54, 58, and 61. In some embodiments, a 5T4 binding agent (e.g., an antibody such as a bispecific antibody or ADC) described herein comprises a VH CDR3 having the amino acid sequence of any one of SEQ ID NOS:3, 9, 15, 20, 29, 33, 37, 41, 48, 52, 55, and 59. In some embodiments, a 5T4 binding agent (e.g., an antibody such as a bispecific antibody or ADC) described herein comprises a VH CDR1 and/or a VH CDR2 and/or a VH CDR3 independently selected from a VH CDR1, VH CDR2, VH CDR3 as depicted in any one of the amino acid sequences depicted in Tables 1-3. In some embodiments, a 5T4 binding agent (e.g., an antibody such as a bispecific antibody or ADC) described herein comprises a VL CDR1 having the amino acid sequence of any one of SEQ ID NOS:4, 10, 16, and 21. In another embodiment, a 5T4 binding agent (e.g., an antibody such as a bispecific antibody or ADC) described herein comprises a VL CDR2 having the amino acid sequence of any one of SEQ ID NOS:5, 11, and 22. In some embodiments, a 5T4 binding agent (e.g., an antibody such as a bispecific antibody or an ADC) described herein comprises a VL CDR3 having the amino acid sequence of any one of SEQ ID NOS:6, 17, 23, 30, 38, 42, 49, 56, and 60. In some embodiments, a 5T4 binding agent (e.g., an antibody such as a bispecific antibody or ADC) described herein comprises a VL CDR1 and/or a VL CDR2 and/or a VL CDR3 independently selected from a VL CDR1, VL CDR2, VL CDR3 as depicted in any one of the amino acid sequences depicted in Tables 1-3.

In some embodiments, a 5T4 binding agent (e.g., an antibody such as a bispecific antibody or ADC) described herein comprises a heavy chain variable (VH) region comprising: (1) a VH CDR1 having an amino acid sequence of selected from the group consisting of: (i) SEQ ID NO:1, 27, or 46, (ii) SEQ ID NO:7, 31, or 50, (iii) SEQ ID NO:12, 34, or 53, (iv) SEQ ID NO:13 or 35, and (v) SEQ ID NO:18, 39, or 57; (2) a VH CDR2 having an amino acid sequence of selected from the group consisting of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 having an amino acid sequence of selected from the group consisting of: (i) SEQ ID NO:3, 29, or 48, (ii) SEQ ID NO:9, 33, or 52, (iii) SEQ ID NO:15, 37, or 55, and (iv) SEQ ID NO:20, 41, or 59; and/or a light chain variable (VL) region comprising: (1) a VL CDR1 having an amino acid sequence of selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO:6, 30, or 49, (ii) SEQ ID NO:17, 38, or 56, and (iii) SEQ ID NO:23, 42, or 60.

In some embodiments, a 5T4 binding agent (e.g., an antibody such as a bispecific antibody or ADC) described herein comprises a heavy chain variable (VH) region comprising: (1) a VH CDR1 having an amino acid sequence of selected from the group consisting of: (i) SEQ ID NO:1, 27, or 46, (ii) SEQ ID NO:7, 31, or 50, (iii) SEQ ID NO:12, 34, or 53, (iv) SEQ ID NO:13 or 35, and (v) SEQ ID NO:18, 39, or 57; (2) a VH CDR2 having an amino acid sequence of selected from the group consisting of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 having an amino acid sequence of selected from the group consisting of: (i) SEQ ID NO:3, 29, or 48, (ii) SEQ ID NO:9, 33, or 52, (iii) SEQ ID NO:15, 37, or 55, and (iv) SEQ ID NO:20, 41, or 59.

In some embodiments, a 5T4 binding agent (e.g., an antibody such as a bispecific antibody or ADC) described herein comprises a light chain variable (VL) region comprising: (1) a VL CDR1 having an amino acid sequence of selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO:6, 30, or 49, (ii) SEQ ID NO:17, 38, or 56, and (iii) SEQ ID NO:23, 42, or 60.

In some embodiments, described herein is an antibody or fragment thereof that binds to 5T4, wherein the antibody or fragment thereof comprises: (a) a heavy chain variable (VH) region comprising: (1) a VH CDR1 having an amino acid sequence of selected from the group consisting of: (i) SEQ ID NO:1, 27, or 46, (ii) SEQ ID NO:7, 31, or 50, (iii) SEQ ID NO:12, 34, or 53, (iv) SEQ ID NO:13 or 35, and (v) SEQ ID NO:18, 39, or 57; (2) a VH CDR2 having an amino acid sequence of selected from the group consisting of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 having an amino acid sequence of selected from the group consisting of: (i) SEQ ID NO:3, 29, or 48, (ii) SEQ ID NO:9, 33, or 52, (iii) SEQ ID NO:15, 37, or 55, and (iv) SEQ ID NO:20, 41, or 59; and/or a light chain variable (VL) region comprising: (1) a VL CDR1 having an amino acid sequence of selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO:6, 30, or 49, (ii) SEQ ID NO:17, 38, or 56, and (iii) SEQ ID NO:23, 42, or 60.

In some embodiments, described herein is an antibody or fragment thereof that binds to 5T4, wherein the antibody or fragment thereof comprises a heavy chain variable (VH) region comprising: (1) a VH CDR1 having an amino acid sequence of selected from the group consisting of: (i) SEQ ID NO:1, 27, or 46, (ii) SEQ ID NO:7, 31, or 50, (iii) SEQ ID NO:12, 34, or 53, (iv) SEQ ID NO:13 or 35, and (v) SEQ ID NO:18, 39, or 57; (2) a VH CDR2 having an amino acid sequence of selected from the group consisting of: (i) SEQ ID NO:2, 28, or 47, (ii) SEQ ID NO:8, 32, or 51, (iii) SEQ ID NO:14, 36, or 54, (iv) SEQ ID NO:19, 40, or 58, and (v) SEQ ID NO:24, 43, or 61; and (3) a VH CDR3 having an amino acid sequence of selected from the group consisting of: (i) SEQ ID NO:3, 29, or 48, (ii) SEQ ID NO:9, 33, or 52, (iii) SEQ ID NO:15, 37, or 55, and (iv) SEQ ID NO:20, 41, or 59.

In some embodiments, described herein is an antibody or fragment thereof that binds to 5T4, wherein the antibody or fragment thereof comprises a light chain variable (VL) region comprising: (1) a VL CDR1 having an amino acid sequence of selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO:6, 30, or 49, (ii) SEQ ID NO:17, 38, or 56, and (iii) SEQ ID NO:23, 42, or 60.

In some embodiments, described herein is an antibody or fragment thereof that binds to 5T4 comprising all three heavy chain complementarity determining regions (CDRs) and/or all three light chain CDRs from: the antibody designated mAbA4 that comprises a VH sequence that is SEQ ID NO:25 and a VL sequence that is SEQ ID NO:26; the antibody designated mAbA15 that comprises a VH sequence that is SEQ ID NO:44 and a VL sequence that is SEQ ID NO:45; or the antibody designated mAbA17 that comprises a VH sequence that is SEQ ID NO:62 and a VL sequence that is SEQ ID NO:63. In some embodiments, the antibody or fragment thereof comprises all three heavy chain CDRs and/or all three light chain CDRs from the antibody designated mAbA4. In some embodiments, the antibody or fragment thereof comprises all three heavy chain CDRs and/or all three light chain CDRs from the antibody designated mAbA15. In some embodiments, the antibody or fragment thereof comprises all three heavy chain CDRs and/or all three light chain CDRs from the antibody designated mAbA17.

In some embodiments, described herein is an antibody or fragment thereof that binds to 5T4, wherein the antibody comprises: (a) a heavy chain variable (VH) region comprising a VH CDR1, a VH CDR2, and a VH CDR3 amino acid sequence depicted in Tables 1-3; and/or (b) a light chain variable (VL) region comprising a VL CDR1, a VL CDR2, and a VL CDR3 amino acid sequence depicted in Tables 1-3. In some embodiments, the antibody comprises a heavy chain variable (VH) region comprising a VH CDR1, a VH CDR2, and a VH CDR3 amino acid sequence depicted in Tables 1-3. In some embodiments, the antibody comprises a light chain variable (VL) region comprising a VL CDR1, a VL CDR2, and a VL CDR3 amino acid sequence depicted in Tables 1-3.

In some embodiments, described herein is an antibody comprising: (a) a heavy chain variable (VH) region comprising: (1) a VH CDR1 having an amino acid sequence selected from the group consisting of SEQ ID NOS:1, 7, 12, 13, and 18; (2) a VH CDR2 having an amino acid sequence selected from the group consisting of SEQ ID NOS:2, 8, 14, 19, and 24; and (3) a VH CDR3 having an amino acid sequence selected from the group consisting of SEQ ID NOS:3, 9, 15, and 20; and (b) a light chain variable (VL) region comprising: (1) a VL CDR1 having an amino acid sequence selected from the group consisting of SEQ ID NOS:4, 10, 16, and 21; (2) a VL CDR2 having an amino acid sequence selected from the group consisting of SEQ ID NOS:5, 11, and 22; and (3) a VL CDR3 having an amino acid sequence selected from the group consisting of SEQ ID NOS:6, 17, and 23.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:7; (2) a VH CDR2 having the amino acid sequence of SEQ ID NO:8; and (3) a VH CDR3 having the amino acid sequence of SEQ ID NO:9; and (b) a light chain variable (VL) region comprising: (1) a VL CDR1 having the amino acid sequence of SEQ ID NO:10; (2) a VL CDR2 having the amino acid sequence of SEQ ID NO:11; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:12; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:13; (2) a VH CDR2 having the amino acid sequence of SEQ ID NO:14; and (3) a VH CDR3 having the amino acid sequence of SEQ ID NO:15; and (b) a light chain variable (VL) region comprising: (1) a VL CDR1 having the amino acid sequence of SEQ ID NO:16; (2) a VL CDR2 having the amino acid sequence of SEQ ID NO:11; and (3) a VL CDR3 having the amino acid sequence of SEQ ID NO:17.

In some embodiments, described herein is an antibody comprising: (a) a heavy chain variable (VH) region comprising: (1) a VH CDR1 having the amino acid sequence of SEQ ID NO:18; (2) a VH CDR2 having the amino acid sequence of SEQ ID NO:19; and (3) a VH CDR3 having the amino acid sequence of SEQ ID NO:20; and (b) a light chain variable (VL) region comprising: (1) a VL CDR1 having the amino acid sequence of SEQ ID NO:21; (2) a VL CDR2 having the amino acid sequence of SEQ ID NO:22; and (3) a VL CDR3 having the amino acid sequence of SEQ ID NO:23.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:24; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having an amino acid sequence selected from the group consisting of SEQ ID NOS:27, 31, 34, 35, and 39; (2) a VH CDR2 having an amino acid sequence selected from the group consisting of SEQ ID NOS:28, 32, 36, 40, and 43; and (3) a VH CDR3 having an amino acid sequence selected from the group consisting of SEQ ID NOS:29, 33, 37, and 41; and (b) a light chain variable (VL) region comprising: (1) a VL CDR1 having an amino acid sequence selected from the group consisting of SEQ ID NOS:4, 10, 16, and 21; (2) a VL CDR2 having an amino acid sequence selected from the group consisting of SEQ ID NOS:5, 11, and 22 and (3) a VL CDR3 having an amino acid sequence selected from the group consisting of SEQ ID NOS:30, 38, and 42.

In some embodiments, described herein is an antibody comprising: (a) a heavy chain variable (VH) region comprising: (1) a VH CDR1 having the amino acid sequence of SEQ ID NO:27; (2) a VH CDR2 having the amino acid sequence of SEQ ID NO:28; and (3) a VH CDR3 having the amino acid sequence of SEQ ID NO:29; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:30.

In some embodiments, described herein is an antibody comprising: (a) a heavy chain variable (VH) region comprising: (1) a VH CDR1 having the amino acid sequence of SEQ ID NO:31; (2) a VH CDR2 having the amino acid sequence of SEQ ID NO:32; and (3) a VH CDR3 having the amino acid sequence of SEQ ID NO:33; and (b) a light chain variable (VL) region comprising: (1) a VL CDR1 having the amino acid sequence of SEQ ID NO:10; (2) a VL CDR2 having the amino acid sequence of SEQ ID NO: 11; and (3) a VL CDR3 having the amino acid sequence of SEQ ID NO:30.

In some embodiments, described herein is an antibody comprising: (a) a heavy chain variable (VH) region comprising: (1) a VH CDR1 having the amino acid sequence of SEQ ID NO:34; (2) a VH CDR2 having the amino acid sequence of SEQ ID NO:28; and (3) a VH CDR3 having the amino acid sequence of SEQ ID NO:29; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:30.

In some embodiments, described herein is an antibody comprising: (a) a heavy chain variable (VH) region comprising: (1) a VH CDR1 having the amino acid sequence of SEQ ID NO:35; (2) a VH CDR2 having the amino acid sequence of SEQ ID NO:36; and (3) a VH CDR3 having the amino acid sequence of SEQ ID NO:37; and (b) a light chain variable (VL) region comprising: (1) a VL CDR1 having the amino acid sequence of SEQ ID NO:16; (2) a VL CDR2 having the amino acid sequence of SEQ ID NO: 11; and (3) a VL CDR3 having the amino acid sequence of SEQ ID NO:38.

In some embodiments, described herein is an antibody comprising: (a) a heavy chain variable (VH) region comprising: (1) a VH CDR1 having the amino acid sequence of SEQ ID NO:39; (2) a VH CDR2 having the amino acid sequence of SEQ ID NO:40; and (3) a VH CDR3 having the amino acid sequence of SEQ ID NO:41; and (b) a light chain variable (VL) region comprising: (1) a VL CDR1 having the amino acid sequence of SEQ ID NO:21; (2) a VL CDR2 having the amino acid sequence of SEQ ID NO:22; and (3) a VL CDR3 having the amino acid sequence of SEQ ID NO:42.

In some embodiments, described herein is an antibody comprising: (a) a heavy chain variable (VH) region comprising: (1) a VH CDR1 having the amino acid sequence of SEQ ID NO:27; (2) a VH CDR2 having the amino acid sequence of SEQ ID NO:43; and (3) a VH CDR3 having the amino acid sequence of SEQ ID NO:29; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:30.

In some embodiments, described herein is an antibody comprising: (a) a heavy chain variable (VH) region comprising: (1) a VH CDR1 having an amino acid sequence selected from the group consisting of SEQ ID NOS:13, 46, 50, 53, and 57; (2) a VH CDR2 having an amino acid sequence selected from the group consisting of SEQ ID NOS:47, 51, 54, 58, and 61; and (3) a VH CDR3 having an amino acid sequence selected from the group consisting of SEQ ID NOS:48, 52, 55, and 59; and (b) a light chain variable (VL) region comprising: (1) a VL CDR1 having an amino acid sequence selected from the group consisting of SEQ ID NOS:4, 10, 16, and 21; (2) a VL CDR2 having an amino acid sequence selected from the group consisting of SEQ ID NOS:5, 11, and 22; and (3) a VL CDR3 having an amino acid sequence selected from the group consisting of SEQ ID NOS:49, 56, and 60.

In some embodiments, described herein is an antibody comprising: (a) a heavy chain variable (VH) region comprising: (1) a VH CDR1 having the amino acid sequence of SEQ ID NO:46; (2) a VH CDR2 having the amino acid sequence of SEQ ID NO:47; and (3) a VH CDR3 having the amino acid sequence of SEQ ID NO:48; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:49.

In some embodiments, described herein is an antibody comprising: (a) a heavy chain variable (VH) region comprising: (1) a VH CDR1 having the amino acid sequence of SEQ ID NO:50; (2) a VH CDR2 having the amino acid sequence of SEQ ID NO:51; and (3) a VH CDR3 having the amino acid sequence of SEQ ID NO:52; and (b) a light chain variable (VL) region comprising: (1) a VL CDR1 having the amino acid sequence of SEQ ID NO:10; (2) a VL CDR2 having the amino acid sequence of SEQ ID NO:11; and (3) a VL CDR3 having the amino acid sequence of SEQ ID NO:49.

In some embodiments, described herein is an antibody comprising: (a) a heavy chain variable (VH) region comprising: (1) a VH CDR1 having the amino acid sequence of SEQ ID NO:53; (2) a VH CDR2 having the amino acid sequence of SEQ ID NO:47; and (3) a VH CDR3 having the amino acid sequence of SEQ ID NO:48; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:49.

In some embodiments, described herein is an antibody comprising: (a) a heavy chain variable (VH) region comprising: (1) a VH CDR1 having the amino acid sequence of SEQ ID NO:13; (2) a VH CDR2 having the amino acid sequence of SEQ ID NO:54; and (3) a VH CDR3 having the amino acid sequence of SEQ ID NO:55; and (b) a light chain variable (VL) region comprising: (1) a VL CDR1 having the amino acid sequence of SEQ ID NO:16; (2) a VL CDR2 having the amino acid sequence of SEQ ID NO:11; and (3) a VL CDR3 having the amino acid sequence of SEQ ID NO:56.

In some embodiments, described herein is an antibody comprising: (a) a heavy chain variable (VH) region comprising: (1) a VH CDR1 having the amino acid sequence of SEQ ID NO:57; (2) a VH CDR2 having the amino acid sequence of SEQ ID NO:58; and (3) a VH CDR3 having the amino acid sequence of SEQ ID NO:59; and (b) a light chain variable (VL) region comprising: (1) a VL CDR1 having the amino acid sequence of SEQ ID NO:21; (2) a VL CDR2 having the amino acid sequence of SEQ ID NO:22; and (3) a VL CDR3 having the amino acid sequence of SEQ ID NO:60.

In some embodiments, described herein is an antibody comprising: (a) a heavy chain variable (VH) region comprising: (1) a VH CDR1 having the amino acid sequence of SEQ ID NO:46; (2) a VH CDR2 having the amino acid sequence of SEQ ID NO:61; and (3) a VH CDR3 having the amino acid sequence of SEQ ID NO:48; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:49.

In some embodiments, described herein is an antibody comprising a VH region and/or VL region described herein, wherein the VH and/or VL comprises human framework sequences. In some embodiments, the VH region and/or VL region comprises a framework 1 (FR1), a framework 2 (FR2), a framework 3 (FR3) and/or a framework 4 (FR4) sequence, such as a human FR1, a human FR2, a human FR3 and/or a human FR4.

In some embodiments, the antibody described herein is a monoclonal antibody. In some embodiments, the monoclonal antibody is a humanized, human or chimeric antibody. In some embodiments, the antibody described herein is a Fab, Fab′, F(ab′)₂, Fv, scFv, (scFv)₂, single chain antibody molecule, dual variable region antibody, single variable region antibody, linear antibody, V region, or multispecific antibody formed from antibody fragments.

In some embodiments, described herein is a binding agent that binds to essentially the same epitope as an antibody or fragment thereof of any one of the antibodies described herein. In some embodiments, described herein is a binding agent that competes for binding to human 5T4 with an antibody or fragment thereof of any one described herein. In some embodiments, the binding agent is an antibody or fragment thereof or an ADC comprising an antibody or fragment thereof.

In certain aspects, the CDRs of a 5T4 binding agent (e.g., an antibody or ADC), including a human 5T4 binding agent, can be determined according to the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In certain aspects, the CDRs of a 5T4 binding agent (e.g., an antibody or ADC), including a human 5T4 binding agent, can be determined according to the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Pat. No. 7,709,226).

In certain aspects, the CDRs of a 5T4 binding agent (e.g., an antibody or ADC), including a human 5T4 binding agent, can be determined according to the ImMunoGeneTics (IMGT®) system, for example, as described in Lefranc, M.-P., 1999, The Immunologist, 7:132-136 and Lefranc, M.-P. et al., 1999, Nucleic Acids Res., 27:209-212 (“IMGT CDRs”).

In certain aspects, the CDRs of a 5T4 binding agent (e.g., an antibody or ADC), including a human 5T4 binding agent, can be determined according to the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001).

In certain aspects, the CDRs of a 5T4 binding agent (e.g., an antibody or ADC), including a human 5T4 binding agent, can be determined according to the Contact system, which will be referred to herein as the “Contact CDRs” (see, e.g., MacCallum R M et al., 1996, J Mol Biol 5: 732-745). The Contact CDRs are based on an analysis of the available complex crystal structures.

In some embodiments, the position of one or more CDRs along the VH (e.g., CDR1, CDR2, or CDR3) and/or VL (e.g., CDR1, CDR2, or CDR3) region of a 5T4 binding agent (e.g., an antibody or ADC), including a human 5T4 binding agent, described herein may vary by one, two, three, four, five, or six amino acid positions so long as binding to 5T4 (e.g., human 5T4) is maintained (e.g., substantially maintained, for example, at least 50%, at least 60%, at least 70%, at least 80%, at least 90%, or at least 95%). For example, in some embodiments, the position defining a CDR of any of Table 1, 2, or 3 may vary by shifting the N-terminal and/or C-terminal boundary of the CDR by one, two, three, four, five, or six amino acids, relative to the current CDR position, so long as binding to 5T4 (e.g., human 5T4) is maintained (e.g., substantially maintained, for example, at least 50%, at least 60%, at least 70%, at least 80%, at least 90%, or at least 95%). In other embodiments, the length of one or more CDRs along the VH (e.g., CDR1, CDR2, or CDR3) and/or VL (e.g., CDR1, CDR2, or CDR3) region of a 5T4 binding agent (e.g., an antibody or ADC), including a human 5T4 binding agent, described herein may vary (e.g., be shorter or longer) by one, two, three, four, five, or more amino acids, so long as binding to 5T4 (e.g., human 5T4) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOS: 1-24, 27-43, or 46-61, so long as binding to 5T4 (e.g., human 5T4) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOS: 1-24, 27-43, or 46-61, so long as binding to 5T4 (e.g., human 5T4) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 described herein may be shortened or extended by one, two, three, four, five or more amino acids compared to one or more of the CDRs described by SEQ ID NOS: 1-24, 27-43, or 46-61, so long as binding to 5T4 (e.g., human 5T4) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 described herein may be shortened or extended by one, two, three, four, five or more amino acids compared to one or more of the CDRs described by SEQ ID NOS: 1-24, 27-43, or 46-61, so long as binding to 5T4 (e.g., human 5T4) is maintained (e.g., substantially maintained, for example, at least 50%, at least 60%, at least 70%, at least 80%, at least 90%, or at least 95%). Any method known in the art can be used to ascertain whether binding to 5T4 (e.g., human 5T4) is maintained, for example, the binding assays and conditions described in the “Examples” section described herein. For example, Example 2 described herein describes an assay for measuring binding to 5T4 (e.g., human 5T4).

In other embodiments, the 5T4 binding agents (e.g., antibodies or ADCs), including human 5T4 binding agents, presented herein that bind to 5T4, further comprise conservative sequence modifications. With respect to polypeptides that are 5T4 binding agents (e.g., antibodies), such as human 5T4 binding agents, conservative sequence modifications include conservative amino acid substitutions that include ones in which the amino acid residue is replaced with an amino acid residue having a similar side chain. Families of amino acid residues having similar side chains have been defined in the art. Thus, in some embodiments, a predicted nonessential amino acid residue in a 5T4 is replaced with another amino acid residue from the same side chain family. Methods of identifying amino acid conservative substitutions which do not eliminate antigen binding and nucleotides encoding thereof are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the conservative sequence modifications described herein modify the amino acid sequences of the 5T4 binding agents (e.g., antibodies or ADCs), including human 5T4 binding agents, by 50%, or 55%, or 60%, or 65%, or 70%, or 75%, or 80%, or 85%, or 90%, or 95%, or 98%, or 99%. In some embodiments, the amino acid sequence modifications refer to at most 1, 2, 3, 4, 5, or 6 amino acid substitutions to the CDRs, such as those described in any one of Tables 1-3. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, a 5T4 binding agent (e.g., an antibody), including a human 5T4 binding agent, contains one or more, including six, CDRs having at least 75%, 80%, 85%, 90%, 95%, 96%, 97%, 98%, or 99% identity to the CDRs of mAbA4, mAbA15, or mAbA17 (see, e.g., Tables 1, 2, or 3). In some embodiments, a 5T4 binding agent (e.g., an antibody), including a human 5T4 binding agent, contains a VH and a VL comprising CDRs identical to those of mAbA4, mAbA15, or mAbA17 (see, e.g., Tables 1, 2, or 3). In some embodiments, the amino acid sequence modifications do not include any modification within an SDR. In some embodiments, the amino acid sequence modifications do not include any modification within a CDR (such as CDR1, CDR2, CDR3, or any combination thereof). In further embodiments, the amino acid sequence modifications are in the framework or constant region.

In some embodiments, provided herein is an antibody or fragment thereof comprising a VH comprising an amino acid sequence having at least 75%, 80%, 85%, 90%, 95%, 96%, 97%, 98%, or 99% identity to SEQ ID NO:25 and a VL comprising an amino acid sequence having at least 75%, 80%, 85%, 90%, 95%, 96%, 97%, 98%, or 99% identity to SEQ ID NO:26, and the binding of the antibody or fragment thereof to 5T4 (e.g., human 5T4) is maintained (e.g., substantially maintained, for example, at least 50%, at least 60%, at least 70%, at least 80%, at least 90%, or at least 95%).

In some embodiments, provided herein is an antibody or fragment thereof comprising a VH comprising an amino acid sequence having at least 75%, 80%, 85%, 90%, 95%, 96%, 97%, 98%, or 99% identity to SEQ ID NO:44 and a VL comprising an amino acid sequence having at least 75%, 80%, 85%, 90%, 95%, 96%, 97%, 98%, or 99% identity to SEQ ID NO:45, and the binding of the antibody or fragment thereof to 5T4 (e.g., human 5T4) is maintained (e.g., substantially maintained, for example, at least 50%, at least 60%, at least 70%, at least 80%, at least 90%, or at least 95%).

In some embodiments, provided herein is an antibody or fragment thereof comprising a VH comprising an amino acid sequence having at least 75%, 80%, 85%, 90%, 95%, 96%, 97%, 98%, or 99% identity to SEQ ID NO:62 and a VL comprising an amino acid sequence having at least 75%, 80%, 85%, 90%, 95%, 96%, 97%, 98%, or 99% identity to SEQ ID NO:63, and the binding of the antibody or fragment thereof to 5T4 (e.g., human 5T4) is maintained (e.g., substantially maintained, for example, at least 50%, at least 60%, at least 70%, at least 80%, at least 90%, or at least 95%).

The present disclosure provides 5T4 binding agents (e.g., antibodies) with a masking moiety and/or cleavable moiety in which one or more of the 5T4 binding domains of the 5T4 binding agent (e.g., antibody) are masked (e.g., via a masking moiety) and/or activatable (e.g., via a cleavable moiety). Technologies for masking of a 5T4 binding agent (e.g., an antibody) are well known in the art, including SAFEbody masking technology (see, e.g., U.S. Patent Application Publication No. 2019/0241886) and Probody masking technology (see, e.g., U.S. Patent Application Publication No. 2015/0079088). Such technologies can be used to generate a 5T4 binding agent (e.g., an antibody) that is masked and/or activatable. Such masked and/or activatable 5T4 binding agents (e.g., antibodies) are useful for the preparation of conjugates, including immunoconjugates, ADCs, masked ADCs and activatable antibody-drug conjugates (AADCs), comprising any one of the 5T4 binding agents (e.g., antibodies), such as human 5T4 binding agents, of the present disclosure, including those directly or indirectly linked another agent such as a drug. For example, 5T4 binding agents (e.g., antibodies), such as human 5T4 binding agents, of the present disclosure may be covalently bound by a synthetic linker to one or more agents such as drugs.

If desired, a 5T4 binding agent (e.g., an antibody), including a human 5T4 binding agent, is linked or conjugated (directly or indirectly) to a moiety with effector function, such as cytotoxic activity (e.g., a chemotherapeutic moiety or a radioisotope) or immune recruitment activity. Moieties that are linked or conjugated (directly or indirectly) include drugs that are cytotoxic (e.g., toxins such as aurostatins) or non-cytotoxic (e.g., signal transduction modulators such as kinases or masking moieties that mask one or more binding domains of a 5T4 binding agent (e.g., antibody), or cleavable moieties that allow for activating a 5T4 binding agent by cleaving of a cleavable moiety to unmask one or more binding domains of a 5T4 binding agent (e.g., antibody) in the tumor microenvironment in the form of masked conjugates. Moieties that promote immune recruitment can include other antigen-binding agents, such as viral proteins that bind selectively to cells of the innate and/or adaptive immune system. Alternatively or in addition, a 5T4 binding agent (e.g., an antibody), including a human 5T4 binding agent, is optionally linked or conjugated (directly or indirectly) to a moiety that facilitates isolation from a mixture (e.g., a tag) or a moiety with reporter activity (e.g., a detection label or reporter protein). It will be appreciated that the features of a 5T4 binding agent (e.g., an antibody), including a human 5T4 binding agent, described herein extend also to a polypeptide comprising a 5T4 binding agent fragment.

In some embodiments, 5T4 binding agents (e.g., antibodies), including 5T4 binding agents, described herein which bind to human 5T4, may be linked or conjugated (directly or indirectly) to a polypeptide, which can result in the generation of an activatable antibody. In some embodiments, a 5T4 binding agent (e.g., an antibody), including a human 5T4 binding agent, is linked or conjugated (directly or indirectly) to an additional agent. In some embodiments, the additional agent is a drug, resulting in an ADC or an AADC when the antibody of the ADC comprises a masking moiety and a cleavable moiety.

In some embodiments, 5T4 binding agents (e.g., antibodies), including human 5T4 binding agents, described herein are conjugated or recombinantly linked (directly or indirectly) to a therapeutic agent (e.g., a cytotoxic agent) or to a diagnostic or detectable agent. The conjugated or recombinantly linked antibodies, including masked or activatable conjugates, can be useful, for example, for treating or preventing a disease, disorder or condition such as a 5T4-mediated disease, disorder or condition. The conjugated or recombinantly linked 5T4 binding agents (e.g., antibodies), including masked or activatable conjugates, can be useful, for example, for monitoring or prognosing the onset, development, progression, and/or severity of a 5T4-mediated disease, disorder or condition.

Such diagnosis and detection can be accomplished, for example, by coupling a 5T4 binding agent (e.g., an antibody) to detectable substances including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

Also described herein are 5T4 binding agents (e.g., antibodies) that are recombinantly linked or conjugated (covalent or non-covalent conjugations, directly or indirectly) to a heterologous protein or polypeptide (or fragment thereof, for example, to a polypeptide (e.g., of about 10, about 20, about 30, about 40, about 50, about 60, about 70, about 80, about 90, or about 100 amino acids) to generate fusion proteins, as well as uses thereof. In particular, described herein are fusion proteins comprising an antigen-binding fragment of a 5T4 binding agent (e.g., an antibody), including a human 5T4 binding agent, described herein (e.g., comprising CDR1, CDR2, and/or CDR3 of VH and/or VL) and a heterologous protein, polypeptide, or peptide. In some embodiments, the heterologous protein, polypeptide, or peptide that a 5T4 binding agent (e.g., an antibody) is linked to is useful for targeting the 5T4 binding agent to a particular cell (e.g., a 5T4-expressing cell, including a tumor cell). Other non-limiting heterologous protein, polypeptide, or peptide that a 5T4 binding agent (e.g., an antibody) is linked to can be useful as an internalization signal or an immune cell engager.

Moreover, 5T4 binding agents (e.g., antibodies), including human 5T4 binding agents, described herein can be linked (directly or indirectly) to marker or “tag” sequences, such as a peptide, to facilitate purification. In some embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In some embodiments, a 5T4 binding agent (e.g., an antibody) described herein is a fusion protein. The term “fusion protein” as used herein refers to a polypeptide that comprises an amino acid sequence of a binding agent (e.g., an antibody) and an amino acid sequence of a heterologous polypeptide or protein (e.g., a polypeptide or protein not normally a part of the antibody). In certain embodiments, the fusion protein retains the biological activity of a 5T4 binding agent. In certain embodiments, the fusion protein comprises a 5T4 antibody VH region, VL region, VH CDR (one, two or three VH CDRs), and/or VL CDR (one, two or three VL CDRs), wherein the fusion protein binds to a 5T4 epitope, a 5T4 fragment and/or a 5T4 polypeptide.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of 5T4 binding agents (e.g., antibodies), including human 5T4 binding agents, as described herein, including, for example, 5T4 binding agents with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). In some embodiments, 5T4 binding agents, including human 5T4 binding agents, may be altered by being subjected to random mutagenesis by error-prone PCR, random nucleotide insertion, or other methods prior to recombination. A polynucleotide encoding a 5T4 binding agent described herein may be recombined with one or more components, motifs, sections, parts, domains, fragments, etc. of one or more heterologous molecules.

5T4 binding agents (e.g., antibodies), including human 5T4 binding agents, described herein may also be attached to solid supports, which are useful for immunoassays or purification of the target antigen. Such solid supports include, but are not limited to, glass, cellulose, polyacrylamide, nylon, polystyrene, polyvinyl chloride, or polypropylene.

5T4 binding agents (e.g., antibodies), including human 5T4 binding agents, described herein can also be linked or conjugated (directly or indirectly) to a second antibody to form an antibody heteroconjugate.

The linker may be a “cleavable moiety” facilitating release of the linked or conjugated agent in a cell, but non-cleavable linkers are also contemplated herein. Linkers for use in conjugates (e.g., ADCs or AADCs) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and agent, including wherein the agent is a drug for the preparation of ADC or an AADC, may be made using a variety of bifunctional protein coupling agents such as N-(β-maleimidopropyloxy)succinimide ester (BMPS); N-ε-malemidocaproyl-oxysuccinimide ester (ECMS); N-γ-malemidocaproyl-oxysuccinimide ester (GMBS); 1,6-hexane-bis-vinylsulfone (HBVS); succinimidyl 4(-N-maleimidomethyl)cyclohexane-1-carboxy-(6-amidocaproate)) (LC-SMCC); m-maleimidobenzoyl-N-hydroxysuccinimide ester (MBS); 4-(4-N-maleimidophenyl)butyric acid hydrazide (MPBH); succinimidil 3-(bromoacetamido) propionate (SBAP); succinimidyl iodoacetate (SIA); succinimidyl (4-iodoacetyl)aminobenzoate (SIAB); succinimidyl-4-(N-maleimidomethyl)cyclohexane-1-carboxylate (SMCC); succinimidyl 4-(p-maleimido-phenyl)butyrate (SMPB); succinimidyl-6-(β-maleimidopropionamido)hexanoate (SMPH); N-(ε-maleimidocaproyloxy)sulfosuccinimide ester (sulfo-ECMS); N-(γ-maleimidobutryloxy)sulfosuccinimide ester (sulfo-GMBS); N-(κ-maleimidoundecanoyloxy)sulfosuccinimide ester (sulfo-KMUS); m-maleimidobenzoyl-N-hydroxysulfosuccinimide ester (sulfo-MBS); sulfosuccinimidyl(4-iodo-acetyl)aminobenzoate (sulfo-SIAB); sulfosuccinimidyl 4-(N-maleimido-methyl)cyclohexane-1-carboxylate (sulfo-SMCC); sulfosuccinimidyl 4-(p-maleimidophenyl)butyrate (sulfo-SMPB); and succinimidyl-(4-vinylsulfone)benzoate) (SVSB).

The present disclosure further contemplates that conjugates of antibodies and agents, including wherein the agent is a drug for the preparation of ADC or AADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of ADC or AADC, have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

In some embodiments, a 5T4 binding agent (e.g., an antibody), including a human 5T4 binding agent, described herein is conjugated to a cytotoxic agent. In some embodiments, a 5T4 binding agent (e.g., an antibody), including a human 5T4 binding agent, disclosed herein can be optionally conjugated with one or more cytotoxic agent(s) disclosed herein or known in the art in order to generate an ADC or AADC. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, a 5T4 binding agent (e.g., an antibody), including a human 5T4 binding agent, described herein is conjugated to a drug such as a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor. In some embodiments, the mitotic inhibitor is a dolastatin, an auristatin, a maytansinoid, and a plant alkaloid. In some embodiments, the drug is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In some embodiments, the anti-tumor antibiotic is selected from the group consisting of an actinomycine, an anthracycline, a calicheamicin, and a duocarmycin. An example of an actinomycine is a pyrrolobenzodiazepine (PBD). An example of an anthracycline is a PNU-anthracycline such as PNU-159682 or derivative.

5T4 binding agents (e.g., antibodies), including human 5T4 binding agents, described herein may be monospecific, bispecific, trispecific or of greater multispecificity. Such agents may include monospecific or multispecific antibodies or ADCs. Multispecific antibodies, such as bispecific antibodies, are monoclonal antibodies that have binding specificities for at least two different targets (e.g., antigens) or two different epitopes on the same target (e.g., a bispecific antibody directed to 5T4 with a first binding domain for a first epitope of a 5T4, and a second binding domain for a second epitope of 5T4). In some embodiments, the monospecific and multispecific (e.g., bispecific) antibodies or ADCs can be constructed based on the sequences of the antibodies described herein, e.g., the CDR sequences listed in Tables 1-3. In some embodiments, the multispecific antibodies described herein are bispecific antibodies. In some embodiments, bispecific antibodies are mouse, chimeric, human or humanized antibodies. In some embodiments, one of the binding specificities of the multispecific antibody is for 5T4 and the other is for any other target (e.g., antigen). In some embodiments, a multispecific (e.g., bispecific) antibody can comprise more than one target (e.g., antigen) binding domain, in which different binding domains are specific for different targets (e.g., a first binding domain that binds to 5T4 and a second binding domain that binds another target (e.g., antigen), such as an immune checkpoint regulator (e.g., a negative checkpoint regulator)). In some embodiments, multispecific (e.g., bispecific) antibody molecules can bind than one (e.g., two or more) epitopes on the same target (e.g., antigen). In some embodiments, one of the binding specificities is 5T4 and the other is for one or more of Cytotoxic T-lymphocyte antigen-4 (CTLA-4), CD80, CD86, Programmed cell death 1 (PD-1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137, CGEN-15001T, CGEN-15022, CGEN-15027, CGEN-15049, CGEN-15052, and CGEN-15092.

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Exemplary structures of multispecific antibodies are known in the art and are further described in Weidle et al., 2013, Cancer Genomics & Proteomics 10: 1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, KA-body, and/or orthogonal Fab.

In some embodiments, BslgG comprises heavy chains that are engineered for heterodimerization. For example, heavy chains can be engineered for heterodimerization using a “knobs-into-holes” strategy, a SEED platform, a common heavy chain (e.g., in KA-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. In some embodiments, an exemplary antibody format is a B-Body format for monospecific or multispecific (e.g., bispecific antibodies) as described in e.g. International Patent Application Publication No. WO 2018/075692 and US Patent Application Publication No. 2018/0118811.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Non-limiting examples of bispecific antibody fragments include, but are not limited to, nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)₂, F(ab′)₂-scFv2, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example, bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be extend the serum half-life of antibody fragments. In embodiments, chemical conjugation, e.g., chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, 5T4 binding agents (e.g., antibodies), including human 5T4 binding agents, disclosed herein can be provided in any antibody format disclosed herein or known in the art. As a non-limiting example, in some embodiments, the 5T4 binding agents (e.g., antibodies), including human 5T4 binding agents, can be selected from Fabs-in-tandem-Ig (FIT-Ig); DVD-Ig; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (Bites; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; Humanized bispecific IgG antibody (REGN1979) (Regeneron); Mab2 bispecific antibodies (F-Star); DVD-Ig=dual variable domain immunoglobulin (Abbott); kappa-lambda bodies; TBTI=tetravalent bispecific tandem Ig; and CrossMab (Roche).

In some embodiments, a multispecific (e.g., bispecific) antibody disclosed herein comprises a 5T4 binding domain and one or more additional binding domains that bind to one or more targets that are not 5T4. In some embodiments, a multispecific (e.g., bispecific) antibody disclosed herein comprises a 5T4 binding domain that comprises the VH and/or VL amino acid sequences as disclosed herein, such as those of Table 1, Table 2, or Table 3.

In some embodiments, described herein is a multispecific (e.g., bispecific) antibody comprising a binding domain which binds to 5T4 that comprises VH and VL CDRs disclosed herein, such as those as set forth in Table 1, Table 2, or Table 3.

Antibodies that bind 5T4 may be obtained by any suitable method, such as (but not limited to) immunization with whole tumor cells comprising 5T4 and collection of antibodies, recombinant techniques, or screening libraries of antibodies or antibody fragments using 5T4 extracellular domain epitopes. Monoclonal antibodies may be generated using a variety of known techniques (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). One exemplary technique for generating monoclonal antibodies comprises immunizing an animal with a human 5T4 antigen and generating a hybridoma from spleen cells taken from the animal. A hybridoma may produce a monoclonal antibody or antibody fragment that binds 5T4.

In a further embodiment, monoclonal antibodies or antibody fragments can be isolated from antibody phage libraries generated using the techniques described in, for example, Antibody Phage Display: Methods and Protocols, P. M. O'Brien and R. Aitken, eds, Humana Press, Totawa N.J., 2002. In principle, synthetic antibody clones are selected by screening phage libraries containing phage that display various fragments of antibody variable region (Fv) fused to phage coat protein. Such phage libraries are screened for against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227: 381-388 (1992).

Screening of the libraries can be accomplished by various techniques known in the art. For example, 5T4 (e.g., a 5T4 polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

5T4 binding agents (e.g., antibodies) can be obtained by designing a suitable antigen screening procedure to select for the phage clone of interest followed by construction of a full length 5T4 binding agent (e.g., an antibody) clone using VH and/or VL sequences (e.g., the Fv sequences), or various CDR sequences from VH and VL sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

Likewise, human antibodies that bind 5T4 may be generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8: 455 58, 1997; Jakobovits et al., Ann. N. Y. Acad. Sci., 764: 525 35, 1995; Green et al., Nature Genet., 7: 13-21, 1994; Lonberg et al., Nature, 368: 856-859, 1994; Taylor et al., Int. Immun. 6: 579-591, 1994; and U.S. Pat. No. 5,877,397.

For example, human antibodies that bind 5T4 may be obtained from transgenic animals that have been engineered to produce specific human antibodies in response to antigenic challenge. For example, International Patent Publication No. WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. International Patent Publication No. WO 96/30498 discloses the use of the Cre/Lox system to modify the immunoglobulin locus in a mammal, such as to replace all or a portion of the constant or variable region to form a modified antibody molecule. International Patent Publication No. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in International Patent Publication No. WO 96/33735. The monoclonal antibodies can be tested for the ability to inhibit or neutralize the biological activity or physiological effect of the corresponding protein.

The present disclosure provides humanized antibodies that bind 5T4, including human 5T4. Humanized antibodies of the present disclosure may comprise one or more CDRs from a VH and/or VL disclosed herein, such as those as shown in Tables 1-3. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind 5T4 may be produced using techniques known to those skilled in the art (Zhang et al., Molecular Immunology, 42(12): 1445-1451, 2005; Hwang et al., Methods, 36(1): 35-42, 2005; Dall'Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567).

In some cases, the humanized antibodies are constructed by CDR grafting, in which the amino acid sequences of the six CDRs of a VH and a VL of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36: 25-34, 2005).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (Sims et al. (1993) J. Immunol. 151:2296; Chothia et al. (1987) J. Mol. Biol. 196:901). Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623). In some cases, the framework is derived from the consensus sequences of the most abundant human subclasses, V_{L}6 subgroup I (V_{L}6I) and V_{H} subgroup III (V_{H}III). In another method, human germline genes are used at the source of the framework regions.

In an alternative paradigm based on comparison of CDRs, called Superhumanization, framework homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as framework donors. Finally, the non-human CDRs are grafted onto these frameworks (see, e.g., Tan et al., J. Immunol. 169: 1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13: 819-824, 2000), Modeller (Sali and Blundell, J. Mol. Biol. 234: 779-815, 1993), and Swiss PDB Viewer (Guex and Peitsch, Electrophoresis 18: 2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, framework residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. (Lazar et al., Mol. Immunol. 44: 1986-1998, 2007).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23: 1105-1116, 2005; Dufner et al., Trends Biotechnol. 24: 523-529, 2006; Feldhaus et al., Nat. Biotechnol. 21: 163-70, 2003; Schlapschy et al., Protein Eng. Des. Sel. 17: 847-60, 2004).

In the framework library approach, a collection of residue variants are introduced at specific positions in the framework followed by selection of the library to select the framework that best supports the grafted CDR. The residues to be substituted may include some or all of the “Vernier” residues identified as potentially contributing to CDR structure (see, e.g., Foote and Winter, J. Mol. Biol. 224: 487-499, 1992), or from the more limited set of target residues identified by Baca et al. (J. Biol. Chem. 272: 10678-10684, 1997).

In framework shuffling, whole frameworks are combined with the non-human CDRs instead of creating combinatorial libraries of selected residue variants (see, e.g., Dall'Acqua et al., Methods 36: 43-60, 2005). The libraries may be screened for binding in a two-step selection process, first humanizing VL, followed by VH. Alternatively, a one-step framework shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44: 3049-60, 2007).

The “humaneering” method is based on experimental identification of essential minimum specificity determinants (MSDs) and is based on sequential replacement of non-human fragments into libraries of human frameworks and assessment of binding. It begins with regions of the CDR3 of non-human VH and VL chains and progressively replaces other regions of the non-human antibody into the human frameworks, including the CDR1 and CDR2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 7: 805-814 (1994), U.S. Pat. Nos. 5,766,886, 5,770,196, 5,821,123, and 5,869,619, and PCT Application Publication WO 93/11794.

In some embodiments, a 5T4 binding agent described herein comprises a non-antibody protein scaffold. Non-limiting examples of such a non-antibody protein scaffold include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate a 5T4 binding agent comprising a non-antibody protein scaffold (see, e.g., Simeon and Chen, Protein Cell, 9(1):3-14 (2018); Yang et al., Annu Rev Anal Chem (Palo Alto Calif). 10(1):293-320 (2017)).

Additionally provided is a nucleic acid encoding a 5T4 binding agent (e.g., antibody or antibody fragment) or a fusion polypeptide as disclosed herein, a nucleic acid complementary thereto, a vector compring a nucleic acid as disclosed herein, a cell comprising a nucleic acid or a vector as disclosed herein. In some embodiments, the cell expresses the 5T4 binding agent. In some embodiments, the cell replicates the nucleic acid or the vector. In some embodiments, provided are the materials for generating 5T4 binding agents, e.g., human 5T4 binding agents, and fragments thereof. For example, an isolated cell may produce a 5T4 binding agent (e.g., antibody or antibody fragment). In this regard, a cell (e.g., an isolated cell) may produce an antibody or fragment thereof comprising a VH and a VL as disclosed herein. In some embodiments, polynucleotides described herein may comprise one or more nucleic acid sequences encoding a 5T4 binding agent (e.g., antibody or antibody fragment). In some embodiments, the polynucleotide is an isolated and/or recombinant polynucleotide. In various aspects, the isolated polynucleotide comprises a nucleotide sequence that encodes a VH and/or a VL, wherein the VH and the VL comprise complementarity determining regions (CDRs) identical to CDRs as disclosed herien.

In some embodiments, one or more vectors (e.g., expression vectors) may comprise one or more polynucleotides for expression of the one or more polynucleotides in a suitable host cell. Such vectors are useful, for example, for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind 5T4 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Expression constructs may also include sequences that facilitate, and preferably promote, homologous recombination in a host cell. In some embodiments, expression constructs of the can also include sequences necessary for replication in a host cell.

Exemplary expression control sequences include promoter/enhancer sequences, e.g., cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132: 2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences whose only intended function is to facilitate large scale production of the vector. One can manufacture and administer polynucleotides for gene therapy using procedures that have been described in the literature for a variety of transgenes. See, e.g., Isner et al., Circulation, 91: 2687-2692, 1995; and Isner et al., Human Gene Therapy, 7: 989-1011, 1996.

In some embodiments, polynucleotides may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

Any suitable vectors may be used to introduce one or more polynucleotides that encode an antibody or fragment thereof into the host. Exemplary vectors that have been described include replication deficient retroviral vectors, including but not limited to lentivirus vectors (Kim et al., J. Virol., 72(1): 811-816, 1998; Kingsman & Johnson, Scrip Magazine, October, 1998, pp. 43-46); parvoviral vectors, such as adeno-associated viral (AAV) vectors (U.S. Pat. Nos. 5,474,935I; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricaudet et al., J. Clin. Invest., 90: 626-630, 1992; and Rosenfeld et al., Cell, 68: 143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

Other non-viral delivery mechanisms contemplated include calcium phosphate precipitation (Graham and Van Der Eb, Virology, 52: 456-467, 1973; Chen and Okayama, Mol. Cell Biol., 7: 2745-2752, 1987; Rippe et al., Mol. Cell Biol., 10: 689-695, 1990) DEAE-dextran (Gopal, Mol. Cell Biol., 5: 1188-1190, 1985), electroporation (Tur-Kaspa et al., Mol. Cell Biol., 6: 716-718, 1986; Potter et al., Proc. Nat. Acad. Sci. USA, 81: 7161-7165, 1984), direct microinjection (Harland and Weintraub, J. Cell Biol., 101: 1094-1099, 1985, DNA-loaded liposomes (Nicolau and Sene, Biochim. Biophys. Acta, 721: 185-190, 1982; Fraley et al., Proc. Natl. Acad. Sci. USA, 76: 3348-3352, 1979; Felgner, Sci Am., 276(6): 102-6, 1997; Felgner, Hum Gene Ther., 7(15): 1791-3, 1996), cell sonication (Fechheimer et al., Proc. Natl. Acad. Sci. USA, 84: 8463-8467, 1987), gene bombardment using high velocity microprojectiles (Yang et al., Proc. Nat. Acad. Sci USA, 87: 9568-9572, 1990), and receptor-mediated transfection (Wu and Wu, J. Biol. Chem., 262: 4429-4432, 1987; Wu and Wu, Biochemistry, 27: 887-892, 1988; Wu and Wu, Adv. Drug Delivery Rev., 12: 159-167, 1993).

A vector (or the antibody or fragment thereof or a nucleic acid as discussed herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814, 1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (Kaneda et al., Science, 243: 375-378, 1989). In some embodiments, the liposome is complexed or employed in conjunction with nuclear nonhistone chromosomal proteins (HMG-1) (Kato et al., J. Biol. Chem., 266: 3361-3364, 1991). In some embodiments, the liposome is complexed or employed in conjunction with both HVJ and HMG-1. Such expression constructs have been successfully employed in transfer and expression of nucleic acid in vitro and in vivo. In some embodiments, a 5T4 binding agent (e.g., an antibody), including a human 5T4 binding agent, is included in the liposome to target the liposome to cells (such as tumor cells) expressing 5T4 on their surface.

A cell may comprise one or more polynucleotides or one or more vectors, e.g., the cell is transformed or transfected with one or more polynucleotides encoding a 5T4 binding agent (e.g., an antibody), including a human 5T4 binding agent, or the one or more vectors comprising the one or more polynucleotides. In some embodiments, cells express a 5T4 binding agent (e.g., an antibody), including a human 5T4 binding agent, containing one or more, including six CDRs having at least 75%, 80%, 85%, 90%, 95%, 96%, 97%, 98%, or 99% identity to the CDRs of mAbA4, mAbA15, or mAbA17 (see, e.g., Tables 1, 2, and/or 3). In some embodiments, the cell expresses a 5T4 binding agent (e.g., an antibody), including a human 5T4 binding agent, containing the VH and the VL comprising CDRs identical to those of mAbA4, mAbA15, and/or mAbA17 (see, e.g., Tables 1, 2, and/or 3). The cells may be prokaryotic cells, such as Escherichia coli (see, e.g., Pluckthun et al., Methods Enzymol., 178: 497-515, 1989), or eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., 5T4 binding agents (e.g., antibodies), including human 5T4 binding agents) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of a 5T4 binding agent (e.g., an antibody) may comprise culturing a host cell and isolating the 5T4 binding agent. Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327: 70-73, 1987). Several devices for accelerating small particles have been developed. One such device relies on a high voltage discharge to generate an electrical current, which in turn provides the motive force (Yang et al., Proc. Natl. Acad. Sci USA, 87: 9568-9572, 1990). The microprojectiles used have consisted of biologically inert substances such as tungsten or gold beads. A host cell may be isolated and/or purified. A host cell also may be a cell transformed in vivo to cause transient or permanent expression of the polypeptide in vivo. A host cell may also be an isolated cell transformed ex vivo and introduced post-transformation, e.g., to produce the polypeptide in vivo for therapeutic purposes. The definition of host cell explicitly excludes a transgenic human being.

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, UK, 1992); and Current Protocols in Molecular Biology, Ausubel ed., Wiley Interscience, New York, 1999).

A 5T4 binding agent (e.g., an antibody), including a human 5T4 binding agent, is produced using any suitable method, e.g., isolated from an immunized animal, recombinantly or synthetically generated, or genetically-engineered, including as described above. Antibody fragments derived from an antibody are obtained by, e.g., proteolytic hydrolysis of an antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1: 422 Academic Press (1967); Nisonoff et al., Arch. Biochem. Biophys., 89: 230-244, 1960; Porter, Biochem. J., 73: 119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1 2.8.10 and 2.10A.1 2.10A.5.

A 5T4 binding agent (e.g., an antibody), including a human 5T4 binding agent, can be genetically engineered. For example, a 5T4 binding agent (e.g., an antibody), including a human 5T4 binding agent, comprises, for example, a variable region domain generated by recombinant DNA engineering techniques. In this regard, a variable region is optionally modified by insertions, deletions, or changes in the amino acid sequence of the antibody to produce an antibody of interest, including as described above. Polynucleotides encoding CDRs of interest are prepared, for example, by using polymerase chain reaction to synthesize variable regions using mRNA of antibody producing cells as a template (see, for example, Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2: 106-110, 1991). Current antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target.

“Humanized antibodies” are antibodies in which CDRs of heavy and light variable chains of non-human immunoglobulins are transferred into a human variable domain. Constant regions need not be present, but if they are, they optionally are substantially identical to human immunoglobulin constant regions, e.g., at least about 85-90%, about 95%, 96%, 97%, 98%, 99% or more identical, in some embodiments. Hence, in some instances, all parts of a humanized immunoglobulin, except possibly the CDRs, are substantially identical to corresponding parts of natural human immunoglobulin sequences. For example, humanized antibodies are human immunoglobulins (e.g., host antibody) in which hypervariable region residues of the host antibody are replaced by hypervariable region residues from a non-human species (donor antibody) such as mouse, rat, rabbit, or a non-human primate having the desired specificity, affinity, and capacity.

In some embodiments, 5T4 binding agents (e.g., antibodies or ADCs) described herein are useful in compositions and in methods of treating, preventing, or alleviating a 5T4-mediated disease, disorder, or condition, including one or more symptoms of the disease, disorder, or condition. 5T4-mediated diseases, disorders, and conditions include cancers, including, but not limited to, any cancer wherein the tumor cells overexpress 5T4.

In some embodiments, described herein is a method for treating tumor immunity in a subject comprising administering to the subject a 5T4 binding agent (e.g., an antibody or ADC) described herein or fragment thereof or a pharmaceutical composition comprising the binding agent (e.g., antibody or ADC) described herein. In some embodiments, provided herein is a 5T4 binding agent (e.g., an antibody or ADC) described herein or fragment thereof or a pharmaceutical composition comprising the binding agent (e.g., antibody or ADC) described herein for use in treating tumor immunity in a subject. In some embodiments, provided herein is use of a 5T4 binding agent (e.g., an antibody or ADC) described herein or fragment thereof or a pharmaceutical composition comprising the binding agent (e.g., antibody or ADC) described herein in the manufacture of a medicament for treating tumor immunity in a subject.

In some embodiments, described herein is a method for treating a cancer or a tumor in a subject comprising administering to the subject a 5T4 binding agent (e.g., an antibody or ADC) described herein or fragment thereof or a pharmaceutical composition comprising the binding agent (e.g., antibody or ADC) described herein. In some embodiments, provided herein is a 5T4 binding agent (e.g., an antibody or ADC) described herein or fragment thereof or a pharmaceutical composition comprising the binding agent (e.g., antibody or ADC) described herein for use in treating a cancer or a tumor in a subject. In some embodiments, provided herein is use of a 5T4 binding agent (e.g., an antibody or ADC) described herein or fragment thereof or a pharmaceutical composition comprising the binding agent (e.g., antibody or ADC) described herein in the manufacture of a medicament for treating a cancer or a tumor in a subject.

In some embodiments, described herein is a method for alleviating one or more symptoms associated with a cancer or a tumor in a subject comprising administering to the subject a 5T4 binding agent (e.g., an antibody or ADC) described herein or fragment thereof or a pharmaceutical composition comprising the binding agent (e.g., antibody or ADC) described herein. In some embodiments, provided herein is a 5T4 binding agent (e.g., an antibody or ADC) described herein or fragment thereof or a pharmaceutical composition comprising the binding agent (e.g., antibody or ADC) described herein for use in alleviating one or more symptoms associated with a cancer or a tumor in a subject. In some embodiments, provided herein is use of a 5T4 binding agent (e.g., an antibody or ADC) described herein or fragment thereof or a pharmaceutical composition comprising the binding agent (e.g., antibody or ADC) described herein in the manufacture of a medicament for alleviating one or more symptoms associated with a cancer or a tumor in a subject.

In some embodiments, described herein is a method for decreasing tumor size in a subject with a tumor comprising administering to the subject a 5T4 binding agent (e.g., an antibody or ADC) described herein or fragment thereof or a pharmaceutical composition comprising the binding agent (e.g., antibody or ADC) described herein. In some embodiments, provided herein is a 5T4 binding agent (e.g., an antibody or ADC) described herein or fragment thereof or a pharmaceutical composition comprising the binding agent (e.g., antibody or ADC) described herein for use in decreasing tumor size in a subject with a tumor. In some embodiments, provided herein is use of a 5T4 binding agent (e.g., an antibody or ADC) described herein or fragment thereof or a pharmaceutical composition comprising the binding agent (e.g., antibody or ADC) described herein in the manufacture of a medicament for decreasing tumor size in a subject with a tumor.

In some embodiments, described herein is a method for enhancing tumor cell removal in a subject with a tumor comprising administering to the subject 5T4 binding agent (e.g., an antibody or ADC) described herein or fragment thereof or a pharmaceutical composition comprising the binding agent (e.g., antibody or ADC) described herein. In some embodiments, provided herein is a 5T4 binding agent (e.g., an antibody or ADC) described herein or fragment thereof or a pharmaceutical composition comprising the binding agent (e.g., antibody or ADC) described herein for use in enhancing tumor cell removal in a subject with a tumor. In some embodiments, provided herein is use of a 5T4 binding agent (e.g., an antibody or ADC) described herein or fragment thereof or a pharmaceutical composition comprising the binding agent (e.g., antibody or ADC) described herein in the manufacture of a medicament for enhancing tumor cell removal in a subject with a tumor.

The subject of a method described herein can be administered one or more therapeutic agents described herein in combination with a 5T4 binding agent (e.g., an antibody or ADC) described herein or fragment thereof or a pharmaceutical composition comprising the binding agent (e.g., antibody or ADC) described herein.

In some embodiments, the antibody is a human antibody, including, but not limited to, an antibody having variable regions in which both the framework and CDR regions are derived from human germline immunoglobulin sequences as described, for example, in Kabat et al. (1991) Sequences of Proteins of Immunological Interest, Fifth Edition, U.S. Department of Health and Human Services, NIH Publication No. 91-3242. If the antibody contains a constant region, the constant region also preferably is derived from human germline immunoglobulin sequences. Human antibodies may comprise amino acid residues not encoded by human germline immunoglobulin sequences, for example, to enhance the activity of the antibody, but do not comprise CDRs derived from other species (e.g., a mouse CDR placed within a human variable framework region).

In some embodiments, cells of the cancer or tumor of a method described herein express 5T4, such as on the cell surface. In further embodiments, cells of the cancer or tumor overexpress 5T4. In some embodiments, a 5T4 binding agent (e.g., an antibody or ADC) is cytotoxic for tumor cells in cell culture. Such cell culture may include tumor cells expressing or overexpressing 5T4. Tumor cells include, but are not limited to, breast cancer cells, bladder cancer cells, melanoma cells, prostate cancer cells, mesothelioma cells, lung cancer cells, testicular cancer cells, thyroid cancer cells, squamous cell carcinoma cells, glioblastoma cells, neuroblastoma cells, uterine cancer cells, colorectal cancer cells, and pancreatic cancer cells.

In some embodiments, described herein is a method of enhancing the removal of tumor cells in a subject. For example, the method comprises administering an amount of a 5T4 binding agent (e.g., an antibody or ADC), such as a human 5T4 binding agent described herein, effective to enhance the removal of the tumor cells. In some embodiments, the method includes administering a 5T4 binding agent (e.g., an antibody or ADC), including a 5T4 binding agent (i) that competes for binding with mAbA4, mAbA15, and/or mAbA17 (see, e.g., CDRs and VH/VL of Tables 1, 2, and/or), (ii) that binds to human 5T4 and/or (iii) that binds the region of a 5T4 recognized by mAbA4, mAbA15, and/or mAbA17 (see, e.g., CDRs and VH/VL of Tables 1, 2, and/or 3), resulting in enhancement of the removal of tumor cells. In some embodiments, one or more binding agents (e.g. antibodies), polynucleotides, vectors, and/or cells as described above can be used in methods of enhancing the removal of tumor cells in vivo (e.g., in a method of treating cancer in a subject).

A method of modulating (e.g., inhibiting, reducing, preventing) tumor growth in a subject also is provided. For example, the method comprises administering to the subject a composition comprising a 5T4 binding agent (e.g., an antibody or ADC) in an amount effective to modulate tumor growth in the subject.

As used herein, “tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated cell growth. Examples of cancers include, but are not limited to: breast cancer, colon cancer, renal cancer, lung cancer, squamous cell myeloid leukemia, hemangiomas, melanomas, astrocytomas, and glioblastomas as well as other cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma}, lymphoma, leukemia, renal cell carcinoma), bladder and urethra (squamous cell carcinoma, transitional cell carcinoma, adenocarcinoma), prostate (adenocarcinoma, sarcoma, small cell carcinoma of the prostate), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma) serous cystadenocarcinoma, mucinous cystadenocarcinoma, unclassified carcinoma, granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma; as well as cancers of the thyroid including medullary thyroid cancer. In some embodiments, the tumor or cancer is a relapsed tumor or cancer. In some embodiments, the tumor or cancer is a metastatic tumor or cancer. In some embodiments, the tumor ro cancer is a primary tumor or cancer. Additionally or alternatively, the tumor or cancer is resistant to a chemotherapy or other non-5T4-target anti-cancer therapy.

Also provided is a method of treating cancer by administering a 5T4 binding agent (e.g., an antibody) such as a human 5T4 binding agent, to a subject in need thereof, alone or in combination with another agent.

“Enhancing” tumor cell removal does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a 5T4 binding agent (e.g., an antibody) of the method.

Additionally, 5T4 binding agents (e.g., antibodies or ADCs) may be used to alleviate or reduce side effects associated with cancer such as, for example, bone deterioration, vertebral collapse, and paralysis. In one aspect, the subject suffers from or is at risk of suffering from bone metastases and a 5T4 binding agent (e.g., an antibody or ADC) is administered in an amount to reduce deterioration of surrounding bone. Accordingly, in some aspects, a 5T4 binding agent prevents bone deterioration due to bone metastases, wherein tumor cell proliferation is or is not reduced. In some aspects, a 5T4 binding agent (e.g., an antibody or ADC) both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen may be inhibited. In contrast, proliferation of metastases located in microenvironments lacking substantial amounts of type 1 collagen may not be inhibited, yet bone deterioration in the vicinity of the metastasis is reduced or prevented.

A particular administration regimen of a 5T4 binding agent (e.g., an antibody or ADC) for a particular subject will depend, in part, upon the agent used, the amount of agent administered, the route of administration, and the cause and extent of any side effects. The amount of agent (e.g., an antibody or ADC) administered to a subject (e.g., a mammal, such as a human) should be sufficient to effect the desired response over a reasonable time frame. According, in some embodiments, the amount of a 5T4 binding agent (e.g., an antibody or ADC) or pharmaceutical composition described herein administered to a subject is an effective amount.

Suitable routes of administering a composition comprising a 5T4 binding agent (e.g., an antibody or ADC), such as a human 5T4 binding agent (e.g., an antibody or ADC), are well known in the art. Although more than one route can be used to administer an agent (e.g., an antibody or ADC), a particular route can provide a more immediate and more effective reaction than another route.

The present disclosure provides a composition, such as pharmaceutical composition, comprising a 5T4 binding agent (e.g., an antibody or ADC) such as a human 5T4 binding agent and a carrier (e.g., a pharmaceutically acceptable carrier). The particular carrier employed may depend on chemico-physical considerations, such as solubility and lack of reactivity with the binding agent or co-therapy, and by the route of administration. Pharmaceutically acceptable carriers are well-known in the art, examples of which are described herein. Illustrative pharmaceutical forms suitable for injectable use include sterile aqueous solutions or dispersions and sterile powders for the extemporaneous preparation of sterile injectable solutions or dispersions. Injectable formulations are further described in, e.g., Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986)). Preparation of pharmaceutical compositions provided herein and their various routes of administration can be carried out in accordance with methods well known in the art. The delivery systems useful in the context of the invention include time-released, delayed release, and sustained release delivery systems such that the delivery of the composition occurs prior to, and with sufficient time to cause, sensitization of the site to be treated. Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids such as sterols such as cholesterol, cholesterol esters, and fatty acids or neutral fats such as mono-di- and triglycerides; hydrogel release systems; sylastic systems; peptide-based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation. A pharmaceutical composition comprising a 5T4 binding agent (e.g., an antibody or ADC) such as a human 5T4 binding agent is, in one aspect, placed within containers, along with packaging material that provides instructions regarding the use of such pharmaceutical compositions. Generally, such instructions include a tangible expression describing the reagent concentration, as well as, in some embodiments, relative amounts of excipient ingredients or diluents (e.g., water, saline or PBS) that may be necessary to reconstitute the pharmaceutical composition.

In some aspects, a method described herein further comprises administering one or more additional agents, including therapeutic agents, which may be present in a composition or may be administered with a 5T4 binding agent (e.g., an antibody or ADC), such as a human 5T4 binding agent, or provided in a separate composition using the same or a different route of administration. The one or more additional agents, including therapeutic agents, may be administered (e.g., for combination therapy) together or separately (e.g., simultaneously, alternatively, sequentially) with a 5T4 binding agent (e.g., antibody or ADC). Such additional therapeutic agents include, but are not limited to, therapeutic antibodies, immunotherapies and immunotherapeutic agents, cytotoxic agents, chemotherapeutic agents, and inhibitors.

Therapeutic antibodies that can be used with a 5T4 binding agent (e.g., an antibody or ADC) as described herein (e.g., for combination therapy) include, but are not limited to, trastuzumab; abciximab; daclizumab; BEC2; IMC-C22; vitaxin; Campath 1H/LDP-03; Smart M195; epratuzumab; bectumomab; visilizumab; CM3, a humanized anti-ICAM3 antibody; IDEC-I 14; ibritumomab tiuxetan; IDEC-131; IDEC-151; IDEC-152; SMART anti-CD3; eculizumab; adalimumab; certolizumab; IDEC-1 51; MDX-CD4; CD20-sreptdavidin; CDP571; LDP-02; OrthoClone OKT4A; ruplizumab; natalizumab; and lerdelimumab; or biosimilars thereof.

Immunotherapies and immunotherapeutic agents that can be used with a 5T4 binding agent (e.g., an antibody or ADC) as described herein (e.g., for combination therapy) include, but are not limited to, cytokines, such as granulocyte-macrophage colony-stimulating factor (GM-CSF), granulocyte-colony stimulating factor (G-CSF), macrophage inflammatory protein (MIP)-I-alpha, interleukins (including IL-I, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IF A); QS-21; DETOX; Levamisole; and Dinitrophenyl (DNP), and combinations thereof, such as, for example, combinations of, interleukins, for example, IL-2 with other cytokines, such as IFN-alpha. In some embodiments, the immunotherapy includes an immunotherapeutic agent that modulates immune responses, for example, a checkpoint inhibitor or a checkpoint agonist. In some embodiments, the immunotherapeutic agent is an antibody modulator that targets PD-1, 5T4, PD-L2, CEACAM (e g., CEACAM-I, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In some embodiments, the immunotherapeutic agent is an agent that increases natural killer (NK) cell activity. In some embodiments, the immunotherapeutic agent is an agent that inhibits suppression of an immune response. In some embodiments, the immunotherapeutic agent is an agent that inhibits suppressor cells or suppressor cell activity. In some embodiments, the immunotherapeutic agent is an agent or therapy that inhibits Treg activity. In some embodiments, the immunotherapeutic agent is an agent that inhibits the activity of inhibitory immune checkpoint receptors.

In some embodiments, the immunotherapeutic agent includes a T or NK or NKT cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, ICOS, SLAM (e.g., SLAMF7), HVEM, LIGHT, CD2, CD27, CD28, CDS, ICAM-1, LFA-I (CD1 la/CDI8), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others) or a bispecific NK cell engager.

Cytotoxic agents that can be used with a 5T4 binding agent (e.g., an antibody or ADC) as described herein (e.g., for combination therapy) include a substance that inhibits or prevents a cellular function and/or causes cell death or destruction. Exemplary cytotoxic agents include, but are not limited to, radioactive isotopes (e.g., At211, I131, I125, Y90, Re186, Re188, Sm153, Bi212, P32, Pb212 and radioactive isotopes of Lu); growth inhibitory agents; enzymes and fragments thereof such as nucleolytic enzymes; and toxins such as small molecule toxins or enzymatically active toxins of bacterial, fungal, plant or animal origin, including fragments and/or variants thereof. Other exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents that can be used with a 5T4 binding agent (e.g., an antibody or ADC) as described herein (e.g., for combination therapy) include chemical compounds useful in the treatment of cancer. Examples of chemotherapeutic agents include, but are not limited to, erlotinib, bortezomib, disulfiram, epigallocatechin gallate, salinosporamide A, carfilzomib, 17-AAG (geldanamycin), radicicol, lactate dehydrogenase A (LDH-A), fulvestrant, sunitib, letrozole, imatinib mesylate, fmasunate, oxaliplatin, 5-FET (5-fluorouracil), leucovorin, Rapamycin, Lapatinib, Lonafamib (SCH 66336), sorafenib, Bayer Labs), gefitinib, AG1478; alkylating agents such as thiotepa and CYTOXAN®; cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; cally statin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5 alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancrati statin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma II and calicheamicin omega I (Angew Chem. Inti. Ed. Engl. 1994 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSKO polysaccharide complex (JHS Natural Products, Eugene, Ore.); razoxane; rhizoxin; sizofuran; spirogermanium; tenuazonic acid; triaziquone; 2,2′,2″-trichlorotriethylamine; trichothecenes especially T-2 toxin, verracurin A, roridin A and anguidine); urethan; vindesine; dacarbazine; mannomustine; mitobronitol; mitolactol; pipobroman; gacytosine; arabinoside “Ara-C”); cyclophosphamide; thiotepa; taxoids, e.g., paclitaxel, ABRAXANE® (Cremophor-free), albumin-engineered nanoparticle formulations of paclitaxel (American Pharmaceutical Partners, Schaumberg, Ill.), and docetaxel/doxetaxel; chloranmbucil; gemcitabine; 6-thioguanine; mercaptopurine; methotrexate; platinum analogs such as cisplatin and carboplatin; vinblastine; etoposide (VP-16); ifosfamide; mitoxantrone; vincristine; vinorelbine; novantrone; teniposide; edatrexate; daunomycin; aminopterin; capecitabine; ibandronate; CPT-1 I; topoisomerase inhibitor RFS 2000; difluorom ethyl ornithine (DMFO); retinoids such as retinoic acid; and pharmaceutically acceptable salts, acids and derivatives of any of the above. Chemotherapeutic agent also includes (i) anti-hormonal agents that act to regulate or inhibit hormone action on tumors such as anti-estrogens and selective estrogen receptor modulators (SERMs), including, for example, tamoxifen (including tamoxifen citrate), raloxifene, droloxifene, iodoxyfene, 4-hydroxytamoxifen, trioxifene, keoxifene, LY117018, onapristone, and toremifme citrate; (ii) aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LEIRTOTECAN®; ABARELIX®; and (ix) pharmaceutically acceptable salts, acids and derivatives of any of the above.

Chemotherapeutic agents also include antibodies, as described above, including alemtuzumab, bevacizumab; cetuximab; panitumumab, rituximab, pertuzumab, tositumomab, and the antibody drug conjugate, gemtuzumab ozogamicin. Additional humanized monoclonal antibodies with therapeutic potential as agents in combination with 5T4 binding agents (e.g., antibodies) as described herein include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nivolumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-8744695, Wyeth Research and Abbott Laboratories) which is a recombinant exclusively human-sequence, full-length IgGI λ antibody genetically modified to recognize interleukin-12 p40 protein. Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-I7-butyrate, hydrocortisone-17-valerate, aclometasone dipropionate, betamethasone valerate, betamethasone dipropionate, prednicarbate, clobetasone-I 7-butyrate, clobetasol-I 7-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNF alpha) blockers such as etanercept, infliximab, adalimumab, certolizumab pegol, golimumab (Simponi), Interleukin 1 (IL-1) blockers such as anakinra, T cell costimulation blockers such as abatacept, Interleukin 6 (IL-6) blockers such as tocilizumab; Interleukin 13 (IL-I3) blockers such as lebrikizumab; Interferon alpha (IFN) blockers such as Rontalizumab; Beta 7 integrin blockers such as rhuMAb Beta7; IgE pathway blockers such as Anti-MI prime; Secreted homotrimeric LTa3 and membrane bound heterotrimer LTal/I32 blockers such as Anti-lymphotoxin alpha (LTa); miscellaneous investigational agents such as thioplatin, PS-341, phenylbutyrate, ET-I8-OCH3, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol); beta-lapachone; lapachol; colchicines; betulinic acid; acetylcamptothecin, scopolectin, and 9-aminocamptothecin); podophyllotoxin; tegafur; bexarotene; bisphosphonates such as clodronate, etidronate, NE-58095, zoledronic acid/zoledronate, alendronate, pamidronate, tiludronate, or risedronate; and epidermal growth factor receptor (EGF-R); vaccines such as THERATOPEO vaccine; perifosine, COX-2 inhibitor (e.g. celecoxib or etoricoxib), proteosome inhibitor (e.g. PS341); CCI-779; tipifamib (R11577); orafenib, ABT510; Bcl-2 inhibitor such as oblimersen sodium pixantrone; farnesyltransferase inhibitors such as lonafamib (SCH 6636); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin combined with 5-FU and leucovorin. Chemotherapeutic agents also include Poly ADP ribose polymerase (PARP) inhibitors: olaparib, rucaprib niraparib, talzoparib.

Inhibitors that can be used with a 5T4 binding agent (e.g., an antibody) as described herein (e.g., for combination therapy) include, but are not limited to, kinase inhibitors such as imatinib, baricitinib gefitinib, erlotinib, sorafenib, dasatinib, sunitinib, lapatinib, nilotinib, pirfenidone, pazopanib, crizotinib, vemurafenib, vandetanib, ruxolitinib, axitinib, bosutinib, regorafenib, tofacitinib, cabozantinib, ponatinib, trametinib, dabrafenib, afatinib, ibrutinib, ceritinib, idelalisib, nintedanib, palbociclib, lenvatinib, cobimetinib, abemaciclib, acalabrutinib, alectinib, binimetinib, brigatinib, encorafenib, erdafitinib, everolimus, fostamatinib, gilter, larotrectinib, lorlatinib, netarsudil, osimertinib, pemigatinib, pexidartinib, ribociclib, temsirolimus, XL-092, XL-147, XL-765, XL-499, and XL-880. In some embodiments, a compound as described herein can be used in combination with a HSP90 inhibitor (e.g., XL888), liver X receptor (LXR) modulators, retinoid-related orphan receptor gamma (RORy) modulators, checkpoint inhibitors such as a CK1 inhibitor or aCK1a inhibitor, a Wnt pathway inhibitor (e.g., SST-215), or a mineralocorticoid receptor inhibitor, (e.g., esaxerenone) or XL-888 for the treatment of a disease disclosed herein such as cancer. In some embodiments, a 5T4 binding agent (e.g., an antibody) as disclosed herein can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, 1NS-R, IGF-1R, IR-R, PDGFαR, PDGFβ/R, CSFIR, KIT, FLK-II, KDR/FLK-1, FLK-4, flt-1, FGFR1, FGFR2, FGFR3, FGFR4, Ron, Sea, TRKA, TRKB, TRKC, FLT3, VEGFR/Flt2, Flt4, EphAI, EphA2, EphA3, EphB2, EphB4, Tie2, Src, Fyn, Lck, Fgr, Btk, Fak, SYR, FRK, JAK (JAK1 and or JAK2), ABL, ALK, CDK7, CDK12, KRAS, and B-Raf.

Additional non-limiting examples of inhibitors that can be used with a 5T4 binding agent (e.g., an antibody or ADC) as described herein, e.g. for treatment of cancer (e.g., for combination therapy) include an FGFR inhibitor (FGFR1, FGFR2, FGFR3 or FGFR4, e.g., pemigatinib, an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, vandetanib, ramucirumab, lenvatinib, ziv-aflibercept), a PARP inhibitor (e.g. olaparib, rucaparib, veliparib or niraparib), a JAK inhibitor (e.g., ruxolitinib, baricitinib, itacitinib), an IDO inhibitor (e.g., epacadostat, NLG919, or BMS-986205, MK7162), an LSD1 inhibitor, a TDO inhibitor, a PI3K-delta inhibitor (e.g., parsaclisib), a PI3K-gamma inhibitor such as PI3K-gamma selective inhibitor, a Pim inhibitor, a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors, or combinations thereof.

In some embodiments, a 5T4 binding agent (e.g., an antibody or ADC) as disclosed herein can be used in combination with inhibitors of PD-1 or inhibitors of 5T4, e.g., an anti-PD-1 monoclonal antibody or an anti-5T4 monoclonal antibody, for example, nivolumab (Opdivo), pembrolizumab (Keytruda, MK-3475), atezolizumab, avelumab, cemiplimab, spartalizumab, camrelizumab, cetrelimab, toripalimab, sintilimab, AB122, JTX-4014, BGB-108, BCD-100, BAT1306, LZM009, AK105, HLX10, and TSR-042, AMP-224, AMP-514, PDR001, durvalumab, pidilizumab (Imfinzi®, CT-011), CK-301, BMS 936559, MPDL3280A, tislelizumab, BMS-935559, MED14736, FAZ053, KN035, CS1001, CBT-502, A167, STI-A101, BGB-A333, MSB-2311, HLX20, AUNP12, CA-170, BMS-986189, LY3300054, and MSB0010718C.

In some embodiments, a multispecific binding agent as disclosed herein can be used in combination with CTLA-4 inhibitors, e.g., an anti-CTLA-4 antibody, for example, ipilimumab (Yervoy), tremelimumab and AGEN1884, or with phosphatidylserine inhibitors, for example, bavituximab (PGN401), or with antibodies to cytokines (IL-10, TGF-b, and the like), or with bispecific antibodies that bind to 5T4 and CTLA-4 (e.g., AK104) or PD-1 and CTLA-4, or with other anti-cancer agents such as cemiplimab.

The additional agent may be a pharmaceutically acceptable salt, ester, amide, hydrate, and/or prodrug of any of these therapeutic agents described above or other agents.

It is understood that modifications which do not substantially affect the activity of the various embodiments described herein are also provided within the definition of the subject matter described herein. Accordingly, the following examples are intended to illustrate but not limit the present disclosure.

### EXAMPLES

### Example 1. Antibody Generation

To obtain binders for human 5T4, antibody discovery was conducted by phage display.

### A. Phage Display

To obtain binders for human 5T4, antibody discovery was conducted by phage display of human Fab libraries and was carried out using standard protocols. The extracellular domain of human 5T4 was purchased from Acro Biosystems (biotinylated human 5T4 His-Avitag Acro Cat No. TPG-H83Eb) or produced. Antigens produced consisted of human 5T4, cynomolgus 5T4, and mouse 5T4, all cloned into a vector containing a C-terminal 6-his-Avi tag and biotinylated in vitro using a co-expressed biotin ligase enzyme using standard procedures where possible.

Phage clones were screened for the ability to bind to biotinylated human 5T4. Briefly, Fab-formatted phage libraries were constructed using expression vectors capable of replication and expression in phage (also referred to as a phagemid). Both the heavy chain and the light chain were encoded in the same expression vector, where the heavy chain was fused to a truncated variant of the phage coat protein pill. The light chain and heavy chain-pill fusion were expressed as separate polypeptides and assembled in the bacterial periplasm, where the redox potential enables disulfide bond formation, to form the antigen binding domain (Fab) of the candidate antibody.

The library was created using sequences derived from a specific human heavy chain variable domain and a specific human light chain variable domain. Light chain variable domains within the screened library were generated with diversity was introduced into the VL CDR3 (L3), and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:72) and light chain (SEQ ID NO:73) scaffolds used in the library are listed below, where “x” represents CDR amino acids that were varied to create the library, and bold italic represents the CDR sequences that were constant.

The sequence for SEQ ID NO:72 was

EVQLVESGGGLVQPGGSLRLSCAASGFTFSXXXXXWVRQAPGKGLEWVA
XXXXXXXXXXXXXXXXXRFTISADTSKNTAYLQMNSLRAEDTAVYYCAR
XXXXXXXXXXXXXXWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSC.

The sequence for SEQ ID NO:73 was

DIQMTQSPSSLSASVGDRVTITCRASQSVSSAVAWYQQKPGKAPKLLIY
SASSLYSGVPSRFSGSRSGTDFTLTISSLQPEDFATYYCXXXXXXXXXF
GQGTKVEIKRTVAAPSVFIFPPSDSQLKSGTASVVCLLNNFYPREAKVQ
WKVDNALQSGNSQESVTEQDSKDSTYSLSSTLTLSKADYEKHKVYACEV
THQGLSSPVTKSFNRGEC.

Diversity was created through mutagenesis using degenerate DNA oligonucleotide primers to introduce diversity into VL CDR3 (L3) and VH CDR1 (H1), CDR2 (H2) and CDR3 (H3) to mimic the diversity found in the natural antibody repertoire, as described in more detail in Kunkel, TA (PNAS Jan. 1, 1985. 82 (2) 488-492), herein incorporated by reference in its entirety. Briefly, uracil-incorporated single-stranded circular DNA were prepared from isolated phage using standard procedures and Kunkel mutagenesis was carried out to introduce diversity to the four CDRs. Chemically-synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to approximately 1×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD600 of approximately 0.6. After 20 minutes incubation at room temperature, infected cells were sub-cultured in 25 mL 2×YT+Ampicillin and M13K07 helper phage (final concentration of approximately 1×10¹⁰ pfu/ml) and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 50 or 100 nM bead-immobilized 5T4 antigen using standard procedures (100 nM 5T4 for round 3, 50 nM 5T4 for round 4). In total, 3-4 rounds of phage panning were performed to enrich in phage displaying Fabs specific for the 5T4 antigen. 5T4-specific enrichment was confirmed using polyclonal ELISA and individual clones were isolated and further verified by performing monoclonal phage ELISA. DNA sequencing was used to determine the sequence of the CDRs of isolated Fab clones containing a candidate antibody.

The genes encoding heavy chain and light chain variable domains of the candidate antibodies were cloned separately into mammalian expression vectors for expression as full length IgGs in mammalian cells.

For the full length IgGs, the heavy chain constant regions (e.g., CH1=regular text; Hinge=italicized text; CH2=bold text; and CH3=underline text) included the following amino acid sequence:

(SEQ ID NO: 74)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

For the full length IgGs, the light chain constant region (e.g., CL) included the following amino acid sequence:

(SEQ ID NO: 75)
RTVAAPSVFIFPPSDSQLKSGTASVVCLLNNFYPREAKVQWKVDNALQS
GNSQESVTEQDSKDSTYSLSSTLTLSKADYEKHKVYACEVTHQGLSSPV
TKSFNRGEC.

The IgG antibodies were purified from culture supernatant using Protein A resin.

### Example 2. Screening Assays

Bivalent binding assays were employed to provide data on whether antibodies with VH/VL obtained by the phage display in Example 1 bound immobilized 5T4 from all three clinically relevant species: human, cynomolgus monkey (cyno), and mouse. For the bivalent binding and cross-reactivity assays the 5T4 human, cyno, and mouse antigens were synthesized using sequences from the protein database and expressed as biotinylated proteins using a his-avi tag and in vivo biotinylation with co-expressed BirA enzyme.

A standard Octet-based assay was used to determine qualitatively if each antibody bound to each antigen. For these assays, 200 nM biotinylated antigens were immobilized on streptavidin biosensors in 1× kinetic buffer (ForteBio). The loaded sensors were then dipped in wells containing an antibody of interest at 500 nM and the association binding signal was monitored. An increase in signal during the association step above the negative control was considered as binding to the antigen.

For qualitative binding affinity using Octet, all 27 antibodies tested showed binding to human 5T4, 13 of the antibodies also showed binding to cyno 5T4, and 13 of the antibodies also showed binding to mouse 5T4. Data for exemplary antibodies are shown in Table 4 below.

TABLE 4
Qualitative bivalent binding affinity by Octet

| | Human 5T4 | Cyno 5T4 | Mouse 5T4 |
|---|---|---|---|
| Antibody | Octet (bivalent) | Octet (bivalent) | Octet (bivalent) |
| mAbA4 | + | + | − |
| mAbA15 | + | + | + |
| mAbA17 | + | + | + |

Monovalent binding assays were employed to determine the relative affinity of each 5T4 binder obtained by the phage display in Example 1 to human 5T4. Human 5T4 was purchased from Acro Biosystems (Acro Catalog No. TPGH52E) and also produced separately. The monovalent interaction of binders to human 5T4 was monitored using an Octet (Pall ForteBio) instrument. For these assays, 36 nM antibody was immobilized on anti-human Fc (AHC) sensors in 1× kinetic buffer (ForteBio). The sensors were then dipped in wells containing a 3× serial dilution of human 5T4 ranging from 1 μM to 1.37 nM. The association was allowed to reach equilibrium. Sensors were then moved to wells containing only 1× kinetic buffer and allowed to dissociate to equilibrium. Association and dissociation rates were measured and from these monovalent K_{D}s were calculated using the onboard analysis software (ForteBio).

Among all 27 tested antibodies, majority of them showed weak binding in the assay of Monovalent Binding Affinity—Internal (Lot 20-019-62). mAbA4, mAbA15, and mAbA17 are among those exhibiting strong or moderate binding in all three lots. The data are shown in Table 5 below.

TABLE 5
Qualitative monovalent binding affinity by Octet

| | Monovalent Binding | Monovalent Binding | Monovalent Binding |
|---|---|---|---|
| | Affinity - Acro TPGH52E5 | Affinity - Internal | Affinity - Acro TPGH52E5 |
| | (Lot B465-87AF1-K2) | (Lot 20-019-62) | (Lot B465-87AF1-JP) |
| | Octet | Octet | Octet |
| Antibody | K_{D} [nM] | K_{D} [nM] | K_{D} [nM] |
| mAbA4 | 0.01 | 83 | 30.2 |
| mAbA15 | 3.62 | 10.4 | 11.2 |
| mAbA17 | 2.81 | 9.34 | 21.1 |

### Example 3: Additional Screening and Selection

Antibodies that were selected for binding to 5T4, for example, such as those described in Example 2, were evaluated for binding to cells that express 5T4.

Cells were harvested at 70-90% confluence on the day of the assay. In a v-bottom 96-well plate (Costar 3897), 100,000 cells per well were incubated with 50 μL per well 8- or 12-point antibody dilution series for 30 minutes at +4° C. After incubation, cells were washed once with PBS by centrifugation at 200×g for 5 minutes. Cells were then incubated with 50 μL per well of Alexa Fluor 488 Goat anti-human IgG Fab (Jackson Immuno Research 109-547-003) at 1:100 dilution for 30 minutes at +4° C. Cells were washed twice with PBS by centrifugation at 200×g for 5 minutes and were acquired on iQue Screener Plus (Sartorius). Mean fluorescence intensities of BL1 (Alexa-Fluor-488) channel were plotted against antibody concentrations to generate a dose response curve and EC50 values.

26 antibodies among 27 tested showed binding affinity to HEK-5T4 cells, 16 antibodies showed binding affinity to MCF-7 cells, and 26 antibodies showed binding affinity to HEK-cyno-5T4 cells.

Antibodies that selected for binding to all three HEK-5T4, MCF-7, and HEK-cyno-5T4 cell lines (including mAbA4, mAbA15, and mAbA17) were further evaluated in internalization assays with HEK-5T4 and MCF-7 cells.

Adherent cells were harvested at 70-90% confluence and were plated overnight at 37° C. in a 96-well black, clear/flat bottom half-area plate (Corning 3882) at 20,000 cells/well in 50 μL culture media. For suspension cell lines, 96-well plates were coated with poly-L-ornithine solution (Millipore-Sigma) for 1 hour at 37° C. and cells were seeded at 40,000 cells per/well density in 50 μL culture media, about 1-2 hours prior to the assay. Antibodies were diluted in cell culture media, mixed with 3× molar concentration of Incucyte human FabFluor-pH red antibody labelling reagent and incubated for 15 minutes at room temperature. 2× final concentration of all dilutions of antibody/FabFluor mix was prepared. 50 μL of 2× antibody/Fab-Fluor mix was then directly added to every well with pre-seeded cells in duplicates and plate was quickly transferred to Incucyte. Plates were then scanned every 30 minutes for up to 24 hours. Care was taken to begin the first scanning (0 hour) within 2-3 min of adding antibody/FabFluor mix to the cells. Plates were analyzed by reading the red signal generated overtime, normalized to the % confluence of cells in that well. Few antibodies were tested at different concentrations on target cell lines, and the best concentration was selected based on a good signal:noise ratio.

In the internalization assays, 6 antibodies (including mAbA4 and mAbA15) showed strong internalization in HEK-5T4 cells, one antibody showed moderate internalization in HEK-5T4 cells, and 7 antibodies showed no internalization or non-specific internalization in HEK-5T4 cells. In additional assays, 2 antibodies showed strong internalization in MCF 7 cells, 4 antibodies showed moderate internalization in HEK-5T4 cells, and 8 antibodies showed no internalization or non-specific internalization in MCF-7 cells.

### Example 4: Developability Assays

Antibodies that were selected for binding to 5T4, for example, such as those described in Examples 2 and 3, were tested in various developability methods. For example, various chromatographic methods, including size exclusion chromatography (SEC), hydrophobic interaction chromatography (HIC), and standup monolayer adsorption chromatography (SMAC) were employed to assess developability factors, such as monomer percentage, solubility, and antibody aggregation or precipitation.

Size exclusion chromatography (SEC) analysis was performed using a 7.8 mm ID×30 cm TSKgel G3000SWXL column (Tosoh Bioscience LLC, PN 08541) with a 6 mm×4 cm guard column (PN 08543) on an Agilent 1100 HPLC. Antibodies were normalized to 1 mg/mL concentration in Dulbecco's PBS (pH 7.4, without Ca²⁺/Mg²⁺) and clarified via centrifugation to pellet particulates while still retaining soluble aggregates. The mobile phase buffer was Dulbecco's PBS (pH 7.4, without Ca²⁺/Mg²⁺) at 2× concentration (diluted from 10× stock concentration. For each sample, 10 μL was loaded and isocratically eluted at 1.0 mL/min over 20 minutes at ambient temperature. Absorbance was monitored at 280 nm. Chromatographic peaks were integrated to determine percentage of homogeneity and retention time. The column stationary phase along with choice of mobile phase supports hydrophobic and electrostatic interactions in addition to molecular sizing (secondary interactions much milder compared to SMAC). Data analysis was performed using Agilent ChemStation B.04.03 SP1.

Results showed that mAbA4, mAbA15, and mAbA17 (among other tested antibodies) had strong developability based on SEC analysis.

Hydrophobic interaction chromatography (HIC) analysis was performed using a 4.6 mm ID×3.5 cm TSKgel Butyl-NPR column (Tosoh Bioscience LLC, PN 14947) on an Agilent 1100 HPLC. Antibodies were normalized to 1 mg/mL concentration in Dulbecco's PBS (pH 7.4, without Ca²⁺/Mg²⁺). The column was equilibrated with 100% mobile phase Buffer A (2 M ammonium sulfate/20 mM sodium phosphate, pH 7.0) at a flow rate of 1 mL/min at ambient temperature. For each sample, 10 μL was loaded and eluted using a gradient from 100% mobile phase buffer A to 100% mobile phase buffer B (20 mM sodium phosphate, pH 7.0) at 1.0 mL/min over 15 minutes, held at 100% B for 3 minutes to wash the column, and returned 100% A for 2 minutes for equilibration. Absorbance was monitored at 280 nm. Sample retention time was calculated and compared to a set of standard controls to identify antibodies with increased retention time (increased hydrophobicity) and the presence of multiple species. Data analysis was performed using Agilent ChemStation B.04.03 SP1.

Results from the hydrophobic interaction chromatography (HIC) analysis showed that certain antibodies had strong developability and others had moderate developability. mAbA4, mAbA15, and mAbA17 are among those having strong developability based on HIC analysis. Antibody hydrophobicity can impact antibody aggregation, solubility and viscosity. Results indicate a low propensity for aggregation and precipitation of these antibodies.

Standup monolayer adsorption chromatography (SMAC) analysis was performed using a 4.6 mm ID×300 mm Zenix SEC 300 column (Sepax Technologies, PN 213300P-4630) with a 4.6 mm×50 mm guard column (PN 213300P-4605) on an Agilent 1100 HPLC. Antibodies were normalized to 1 mg/mL concentration in Dulbecco's PBS (pH 7.4, without Ca2+/Mg2+) and clarified via centrifugation to pellet particulates while still retaining soluble aggregates. The mobile phase buffer was Dulbecco's PBS (pH 7.4, without Ca2+/Mg2+) at 2× concentration (diluted from 10× stock concentration). For each sample, 10 μL was loaded and isocratically eluted at 0.4 mL/min over 32 minutes at ambient temperature. Absorbance was monitored at 280 nm. Sample retention time was calculated and compared to a set of standard controls to identify antibodies with increased retention time (increased propensity to form aggregates and/or increased hydrophobic/electrostatic interactions) as the column stationary phase along with mobile phase selection promotes secondary interactions in addition to molecular sizing. Data analysis was performed using Agilent ChemStation B.04.03 SP1.

Results from the standup monolayer adsorption chromatography (SMAC) analysis showed that certain antibodies had strong developability and others had moderate developability. mAbA4, mAbA15, and mAbA17 are among those having strong developability based on SMAC analysis. The determination was based on good retention times, which indicate colloidal stability and low propensity to aggregate.

### Example 5: Epitope Binning Assays

Epitope binning assays were employed to determine which epitopes of 5T4 each antibody obtained by phage display in Example 1 bound to.

In order to maintain overall structure of the 5T4 protein while still enabling epitope binding determination, mouse-human chimeras were generated by cloning each of seven contiguous sequences of human 5T4 (Epitopes 1-7) in to replace each of the corresponding mouse sequences. For chimera generation, the full-length human and mouse 5T4 genes were synthesized by GenScript and cloned into a mammalian vector with a 6-His-Avi tag. Each of the seven sequences (Epitopes 1-7) as described herein were then removed from the human gene and separately cloned into the mouse gene using appropriate primers. Epitope 1 of human 5T4 (H1) corresponds to SSPTSSASSFSSSAPFLASAVSAQPPLPDQCPALCECSEAART (SEQ ID NO:76); Epitope 2 of human 5T4 (H2) corresponds to VKCVNRNLTEVPTDLPAYVRNLFLTGNQLAVLPAGAFARRPPLAELAALNLSGSRLDE VR (SEQ ID NO:77); Epitope 3 of human 5T4 (H3) corresponds to AGAFEHLPSLRQLDLSHNPLADLSPFAFSG (SEQ ID NO:78); Epitope 4 of human 5T4 (H4) corresponds to SNASVSAPSPLVELILNHIVPPEDERQNRSFEGMVVAALLAGRALQGLRRLE (SEQ ID NO:79); Epitope 5 of human 5T4 (H5) corresponds to LASNHFLYLPRDVLAQLPSLRHLDLSNNSLVSLTYVSFRNLTHLESL (SEQ ID NO:80); Epitope 6 of human 5T4 (H6) corresponds to HLEDNALKVLHNGTLAELQGLPHIRVFL (SEQ ID NO:81); and Epitope 7 of human 5T4 (H7) corresponds to DNNPWVCDCHMADMVTWLKETEVVQGKDRLTCAYPEKMRNRVLLELNSADLDCDPI LPPSLQTS (SEQ ID NO:82). Antigens were transiently expressed using the Expi293 protein expression system (Thermo Fisher) and biotinylated in vitro using coexpressed BirA biotin ligase and purified using Ni-NTA affinity chromatography. As shown in Table 6, the seven chimeric constructs of human (H) and mouse (M) sequences generated were: (1) H1 M2 M3 M4 M5 M6 M7 (SEQ ID NO:83); (2) M1 H2 M3 M4 M5 M6 M7 (SEQ ID NO:84); (3) M1 M2 H3 M4 M5 M6 M7 (SEQ ID NO:85); (4) M1 M2 M3 H4 M5 M6 M7 (SEQ ID NO:86); (5) M1 M2 M3 M4 H5 M6 M7 (SEQ ID NO:87); (6) M1 M2 M3 M4 M5 H6 M7 (SEQ ID NO:88); and (7) M1 M2 M3 M4 M5 M6 H7 (SEQ ID NO:89). Each antibody was then tested for binding to each of the chimeric constructs and full-length human protein. Any significant binding signal to a chimera indicates the region to which the antibody specifically binds.

TABLE 6
5T4 Chimeric Constructs

| | | |
|---|---|---|
| Human | H1 M2 M3 M4 M5 | SSPTSSASS FSSSAPFLAS AVSAQPPLPD QCPALCECSE |
| 5T4 | M6 M7 | AARTVKCVNR NLLEVPADLP PYVRNLFLTG NQMTVLPAGA |
| Epitope 1 | (SEQ ID NO: 83) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 H2 M3 M4 M5 | SAPSSSVPS NLTEVPTDLP AYVRNLFLTG NQLAVLPAGA |
| 5T4 | M6 M7 | AARTVKCVNR SSTSPAAFLA SGSAQPPPAE RCPAACECSE |
| Epitope 2 | (SEQ ID NO: 84) | FARRPPLAEL AALNLSGSRL DEVRAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 M2 H3 M4 M5 | SAPSSSVPS SSTSPAAFLA SGSAQPPPAE RCPAACECSE |
| 5T4 | M6 M7 | AARTVKCVNR NLLEVPADLP PYVRNLFLTG NQMTVLPAGA |
| Epitope 3 | (SEQ ID NO: 85) | FARQPPLADL EALNLSGNHL KEVCAGAFEH LPSLRQLDLS |
| | | HNPLADLSPF AFSGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 M2 M3 H4 M5 | SAPSSSVPS SSTSPAAFLA SGSAQPPPAE RCPAACECSE |
| 5T4 | M6 M7 | AARTVKCVNR NLLEVPADLP PYVRNLFLTG NQMTVLPAGA |
| Epitope 4 | (SEQ ID NO: 86) | FARQPPLADL EALNLSGNHL KEVCAGAFEH LPGLRRLDLS |
| | | HNPLTNLSAF AFAGSNASVS APSPLVELIL NHIVPPEDER |
| | | QNRSFEGMVV AALLAGRALQ GLRRLELASN HFLFLPRDLL |
| | | AQLPSLRYLD LRNNSLVSLT YASFRNLTHL ESLHLEDNAL |
| | | KVLHNSTLAE WHGLAHVKVF LDNNPWVCDC YMADMVAWLK |
| | | ETEVVPDKAR LTCAFPEKMR NRGLLDLNSS DLDCDAVLPQ |
| | | SLQTS |
| Human | M1 M2 M3 M4 H5 | SAPSSSVPS SSTSPAAFLA SGSAQPPPAE RCPAACECSE |
| 5T4 | M6 M7 | AARTVKCVNR NLLEVPADLP PYVRNLFLTG NQMTVLPAGA |
| Epitope 5 | (SEQ ID NO: 87) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLY |
| | | LPRDVLAQLP SLRHLDLSNN SLVSLTYVSF RNLTHLESLH |
| | | LEDNALKVLH NSTLAEWHGL AHVKVFLDNN PWVCDCYMAD |
| | | MVAWLKETEV VPDKARLTCA FPEKMRNRGL LDLNSSDLDC |
| | | DAVLPQSLQT S |
| Human | M1 M2 M3 M4 M5 | SAPSSSVPS SSTSPAAFLA SGSAQPPPAE RCPAACECSE |
| 5T4 | H6 M7 | AARTVKCVNR NLLEVPADLP PYVRNLFLTG NQMTVLPAGA |
| Epitope 6 | (SEQ ID NO: 88) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NGTLAELQGL PHIRVFLDNN PWVCDCYMAD |
| | | MVAWLKETEV VPDKARLTCA FPEKMRNRGL LDLNSSDLDC |
| | | DAVLPQSLQT S |
| Human | M1 M2 M3 M4 M5 | SAPSSSVPS SSTSPAAFLA SGSAQPPPAE RCPAACECSE |
| 5T4 | M6 H7 | AARTVKCVNR NLLEVPADLP PYVRNLFLTG NQMTVLPAGA |
| Epitope 7 | (SEQ ID NO: 89) | FARQPPLADL EALNLSGNHL KEVCAGAFEH LPGLRRLDLS |
| | | HNPLTNLSAF AFAGSNASVS APSPLEELIL NHIVPPEDQR |
| | | QNGSFEGMVA FEGMVAAALR SGLALRGLTR LELASNHFLF |
| | | LPRDLLAQLP SLRYLDLRNN SLVSLTYASF RNLTHLESLH |
| | | LEDNALKVLH NSTLAEWHGL AHVKVFLDNN PWVCDCHMAD |
| | | MVTWLKETEV VQGKDRLTCA YPEKMRNRVL LELNSADLDC |
| | | DPILPPSLQT S |

A standard Octet-based assay was used to for the epitope binning assay to establish an epitope bin for each antibody. For these assays, 50 nM biotinylated antigens were immobilized on streptavidin sensors in 1× kinetic buffer (ForteBio). The sensors were then dipped in wells containing the antibody in question at 100 nM and the association signal was monitored. The chimera(s) which effectively knocked out binding of an antibody then corresponds to the specific epitope(s) to which the antibody binds. Full-length human 5T4 was included in the assay for reference.

The results show that 9 antibodies specifically bind to Epitope 4 of 5T4, 3 antibodies specifically bind to Epitope 7 of 5T4, and 3 antibodies specifically bind to Epitope 2 of 5T4, one antibody specifically binds to both Epitope 4 and Epitope 6 of 5T4, and 11 antibodies bind to full length 5T4 (FL). Exemplary epitope binning results (for mAbA4, mAbA15 and mAbA17) are summarized in Table 7.

TABLE 7
Epitope Binning Assays

| Antibody | Epitope Bin |
|---|---|
| mAbA4 | 4 |
| mAbA15 | 2 |
| mAbA17 | 7 |

### Example 6: Antibody Selection

As described above, among all the antibodies screened from the phage library as described in Example 1, 27 antibodies that were still able to bind to 5T4 after formatting into IgG were further tested as described in Examples 2-5. Three antibodies designated mAbA4, mAbA15 and mAbA17 were selected based on a multiplicity of activities, including their specificity for human 5T4 binding, their binding to cyno 5T4, their cell surface binding signal, and their strong developability as assessed by a variety of methods such as SEC, HIC, and SMAC. The VH, VL, and CDR sequences of these antibodies are shown in Tables 1-3.

### Example 7. Binding of the Antibodies to Human 5T4 (ELISA)

### A. Normal Binding

The selected antibodies were tested for their ability to bind to hu5T4 using ELISA. On Day 1, unconjugated human TPBG/5T4 protein with a c-terminal polyhistidine and Avi (AVITAG™) tag (AcroBiosystems (Newark, DE, USA), Cat. No. TPG-H52E5) was diluted 2 μg/mL in PBS, mixed thoroughly, plated 100 μL per well in a 96-well plate (Nunc MAXISORP®) and incubated overnight at 4° C.

On Day 2, plates were washed four times with 0.1% Tween®-20 in PBS (“PBS-T”). Wells were blocked with 200 μL of casein in PBS (Blocker™ Casein, available from ThermoFisher Scientific) and incubated for 2 hours at room temperature while shaking.

A 1:3 dilution series of IgG-ADC was prepared in PBS and added at a volume of 100 μL per well in a 96-well plate. Plates were incubated for 1 hour at room temperature while shaking.

Goat anti-human IgG FC gamma specific HRP conjugate (Jackson Immunoresearch, cat #109-035-098) was diluted to a factor of 1:15000 in PBS. ELISA plates were washed six times with PBS-T with a plate washer (BioTek ELx405) after which 100 μL of the goat anti-human IgG FC gamma specific HRP conjugate was added into each well. Plates were then incubated for 30 minutes at room temperature while shaking.

ELISA plates were washed six times with PBS-T using a plate washer (BioTek ELx405) after which 100 μL of 3,3′,5,5′-tetramethylbenzidine (TMB) was added to each well. Plates were allowed to develop until a dark blue color became visible. H₂SO₄ (2 N, 100 μL) was then added to quench the reaction and absorbance of each well was read on a plate reader (SpetraMax M5 equipped with SoftMaxPro software available from Molecular Devices).

### B. Stress Testing

1. High pH Stress Test. The antibodies were tested for their ability to bind to 5T4 using ELISA under basic (high pH) conditions. 1 mg/mL of antibody in 0.1% w/w H₂O₂/1× PBS was stored at room temperature for 24-48 hours in the dark after which 50 mM methionine was added. Samples were frozen for storage prior to analysis, which was carried out via ELISA as described in part A, above. Table 8 below reports the EC₅₀ calculated for each antibody tested.

TABLE 8
Binding of the Antibodies to hu5T4 under High pH

| mAb | condition | EC₅₀ (nM) |
|---|---|---|
| mAbA4 | pH 8.5 Day 0 | 0.120 |
| mAbA4 | pH 8.5 Day 1 | 0.127 |
| mAbA4 | pH 8.5 Day 4 | 0.114 |
| mAbA4 | pH 8.5 Day 7 | 0.128 |
| mAbA4 | pH 8.5 Day 14 | 0.136 |
| mAbA15 | pH 8.5 Day 0 | 0.134 |
| mAbA15 | pH 8.5 Day 1 | 0.128 |
| mAbA15 | pH 8.5 Day 4 | 0.133 |
| mAbA15 | pH 8.5 Day 7 | 0.143 |
| mAbA15 | pH 8.5 Day 14 | 0.131 |
| mAbA17 | pH 8.5 Day 0 | 0.153 |
| mAbA17 | pH 8.5 Day 1 | 0.174 |
| mAbA17 | pH 8.5 Day 4 | 0.150 |
| mAbA17 | pH 8.5 Day 7 | 0.158 |
| mAbA17 | pH 8.5 Day 14 | 0.167 |

2. Low pH Stress Test. The antibodies were also tested for their ability to bind to 5T4 using ELISA under acidic (low pH) conditions. Solutions of 1 mg/mL to 10 mg/mL of antibody in 20 mM Tris/10 mM EDTA at a pH of 8.5 were prepared and stored at 37° C. for 1 to 2 weeks. Samples were frozen for storage prior to analysis, which was carried out via ELISA as described in part A, above. Table 9 below reports the EC₅₀ calculated for each antibody tested.

TABLE 9
Binding of the Antibodies to hu5T4 under Low pH

| mAb | condition | EC₅₀ (nM) |
|---|---|---|
| mAbA4 | pH 5.5 Day 0 | 0.133 |
| mAbA4 | pH 5.5 Day 1 | 0.132 |
| mAbA4 | pH 5.5 Day 4 | 0.125 |
| mAbA4 | pH 5.5 Day 7 | 0.124 |
| mAbA4 | pH 5.5 Day 14 | 0.122 |
| mAbA15 | pH 5.5 Day 0 | 0.124 |
| mAbA15 | pH 5.5 Day 1 | 0.134 |
| mAbA15 | pH 5.5 Day 4 | 0.123 |
| mAbA15 | PH 5.5 Day 7 | 0.132 |
| mAbA15 | pH 5.5 Day 14 | 0.138 |
| mAbA17 | pH 5.5 Day 0 | 0.164 |
| mAbA17 | pH 5.5 Day 1 | 0.159 |
| mAbA17 | pH 5.5 Day 4 | 0.157 |
| mAbA17 | pH 5.5 Day 7 | 0.190 |
| mAbA17 | pH 5.5 Day 14 | 0.153 |

3. Oxidative Stress. The antibodies were tested for their ability to bind to 5T4 using ELISA in an oxidative environment. Solutions of 1 mg/mL to 10 mg/mL of antibody in 20 mM citrate/50 mM NaCl at a pH of 5.5 were prepared and stored at 37° C. for 1 to 2 weeks. Samples were frozen for storage prior to analysis, which was carried out via ELISA as described in part A, above. Table 10 below reports the EC₅₀ calculated for each antibody tested.

TABLE 10
Binding of the Antibodies to hu5T4 under Oxidative Stress

| mAb | condition | EC₅₀ (nM) |
|---|---|---|
| mAbA4 | PBS Time 0 | 0.182 |
| mAbA4 | PBS time 24 hr | 0.200 |
| mAbA4 | PBS time 48 hr | 0.189 |
| mAbA4 | PBS + H₂O₂ time 0 | 0.225 |
| mAbA4 | PBS + H₂O₂ time 24 hr | 0.393 |
| mAbA4 | PBS + H₂O₂ time 48 hr | 0.386 |
| mAbA15 | PBS Time 0 | 0.229 |
| mAbA15 | PBS time 24 hr | 0.212 |
| mAbA15 | PBS time 48 hr | 0.203 |
| mAbA15 | PBS + H₂O₂ time 0 | 0.191 |
| mAbA15 | PBS + H₂O₂ time 24 hr | 0.190 |
| mAbA15 | PBS + H₂O₂ time 48 hr | 0.189 |
| mAbA17 | PBS Time 0 | 0.198 |
| mAbA17 | PBS time 24 hr | 0.186 |
| mAbA17 | PBS time 48 hr | 0.185 |
| mAbA17 | PBS + H₂O₂ time 0 | 0.188 |
| mAbA17 | PBS + H₂O₂ time 24 hr | 0.183 |
| mAbA17 | PBS + H₂O₂ time 48 hr | 0.207 |

### Example 8. Crossreactivity of the Antibodies to Cyno, Rat, and Mouse 5T4 (ELISA & OCTET)

The selected antibodies were tested for their ability to bind to non-human 5T4-Ts using ELISA and Octet. The ELISA protocol described in Example 7 was used, replacing the human 5T4 antigen for recombinant cyno 5T4-Fc (sourced from either R&D Systems (Minneapolis, MN, USA), Cat. No. 2280-TG-100 or LSBio (Seattle, WA, USA), Cat. No. LS-G137131), recombinant mouse 5T4-Fc (R&D Systems, Cat. No. 5049-TG-100), Recombinant Rat TPBG/5T4 with 6His-SUMO, N-terminus tags (LSBio Cat. No. G56592), Recombinant Mouse TPBG/5T4 with His, N-terminal tag (LSBio, Cat. No. G12168) antigen. For Octet analyses, a Protein A Biochip was coated with the antibody, washed with PBS, and then reacted with the antigen, as identified above. All antibodies tested bound to human, cyno, and rat 5T4. No significant binding crossreactivity to mouse 5T4 was observed.

### Example 9. Cell Binding of the Antibodies to 5T4-Transfected CHO and HEK293 Cells

The selected antibodies were tested for their ability to bind to HEK293 and CHO cells transfected to express human, cyno, rat, or mouse 5T4. Non-transfected cells were utilized as a control to observe non-specific binding. mAbA4, mAbA15, and mAbA17 were reactive to HEK293 and CHO cells transfected to express human and cyno 5T4 but not to cells transfected to express rat or mouse 5T4.

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

What is claimed is:

1. An antibody or fragment thereof that binds to 5T4, wherein the antibody or fragment thereof comprises
(i) a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26;
(ii) a VH CDR1, a VH CDR2, and a VH CDR3 as set forth in a VH comprising the amino acid sequence of SEQ ID NO:44 and a VL CDR1, a VL CDR2, and a VL CDR3 as set forth in a VL comprising the amino acid sequence of SEQ ID NO:45; or
(iii) a VH CDR1, a VH CDR2, and a VH CDR3 as set forth in a VH comprising the amino acid sequence of SEQ ID NO:62 and a VL CDR1, a VL CDR2, and a VL CDR3 as set forth in a VL comprising the amino acid sequence of SEQ ID NO:63.

2. An antibody or fragment thereof that binds to 5T4, wherein the antibody or fragment thereof comprises
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 2, 8, 14, 19, and 24; and
(3) a VH CDR3 having an amino acid sequence selected from the group consisting of SEQ ID NOs: 3, 9, 15, and 20;
and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 6, 17, and 23.

3. An antibody or fragment thereof that binds to 5T4, wherein the antibody or fragment thereof comprises:
(i) a VH region comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6;
(ii) a VH region comprising a VH CDR1 comprising the amino acid sequence of SEQ ID NO:7, a VH CDR2 comprising the amino acid sequence of SEQ ID NO:8, a VH CDR3 comprising the amino acid sequence of SEQ ID NO:9; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:6;
(iii) a VH region comprising a VH CDR1 comprising the amino acid sequence of SEQ ID NO:12, a VH CDR2 comprising the amino acid sequence of SEQ ID NO:2, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6;
(iv) a VH region comprising a VH CDR1 comprising the amino acid sequence of SEQ ID NO:13, a VH CDR2 comprising the amino acid sequence of SEQ ID NO:14, a VH CDR3 comprising the amino acid sequence of SEQ ID NO:15; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:17;
(v) a VH region comprising a VH CDR1 comprising the amino acid sequence of SEQ ID NO:18, a VH CDR2 comprising the amino acid sequence of SEQ ID NO:19, a VH CDR3 comprising the amino acid sequence of SEQ ID NO:20; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:23; or
(vi) a VH region comprising a VH CDR1 comprising the amino acid sequence of SEQ ID NO:1, a VH CDR2 comprising the amino acid sequence of SEQ ID NO:24, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

4. An antibody or fragment thereof that binds to 5T4, wherein the antibody or fragment thereof comprises
(a) a VH region comprising:
(1) a VH CDR1 having an amino acid sequence selected from the group consisting of SEQ ID NOs: 27, 31, 34, 35, and 39;
(2) a VH CDR2 having an amino acid sequence selected from the group consisting of SEQ ID NOs: 28, 32, 36, 40, and 43; and
(3) a VH CDR3 having an amino acid sequence selected from the group consisting of SEQ ID NOs: 29, 33, 37, and 41;
and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 30, 38, and 42.

5. An antibody or fragment thereof that binds to 5T4, wherein the antibody or fragment thereof comprises:
(i) a VH region comprising a VH CDR1 comprising the amino acid sequence of SEQ ID NO:27, a VH CDR2 comprising the amino acid sequence of SEQ ID NO:28, a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30;
(ii) a VH region comprising a VH CDR1 comprising the amino acid sequence of SEQ ID NO:31, a VH CDR2 comprising the amino acid sequence of SEQ ID NO:32, a VH CDR3 comprising the amino acid sequence of SEQ ID NO:33; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:30;
(iii) a VH region comprising a VH CDR1 comprising the amino acid sequence of SEQ ID NO:34, a VH CDR2 comprising the amino acid sequence of SEQ ID NO:28, a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30;
(iv) a VH region comprising a VH CDR1 comprising the amino acid sequence of SEQ ID NO:35, a VH CDR2 comprising the amino acid sequence of SEQ ID NO:36, a VH CDR3 comprising the amino acid sequence of SEQ ID NO:37; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:38;
(v) a VH region comprising a VH CDR1 comprising the amino acid sequence of SEQ ID NO:39, a VH CDR2 comprising the amino acid sequence of SEQ ID NO:40, a VH CDR3 comprising the amino acid sequence of SEQ ID NO:41; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:42; or
(vi) a VH region comprising a VH CDR1 comprising the amino acid sequence of SEQ ID NO:27, a VH CDR2 comprising the amino acid sequence of SEQ ID NO:43, a VH CDR3 comprising the amino acid sequence of SEQ ID NO:29; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:30.

6. An antibody or fragment thereof that binds to 5T4, wherein the antibody or fragment thereof comprises
(a) a VH region comprising:
(1) a VH CDR1 having an amino acid sequence selected from the group consisting of SEQ ID NOs: 46, 50, 53, 13, and 57;
(2) a VH CDR2 having an amino acid sequence selected from the group consisting of SEQ ID NOs: 47, 51, 54, 58, and 61; and
(3) a VH CDR3 having an amino acid sequence selected from the group consisting of SEQ ID NOs: 48, 52, 55, and 59;
and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 49, 56, and 60.

7. An antibody or fragment thereof that binds to 5T4, wherein the antibody or fragment thereof comprises
(i) a VH region comprising a VH CDR1 comprising the amino acid sequence of SEQ ID NO:46, a VH CDR2 comprising the amino acid sequence of SEQ ID NO:47, a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49;
(ii) a VH region comprising a VH CDR1 comprising the amino acid sequence of SEQ ID NO:50, a VH CDR2 comprising the amino acid sequence of SEQ ID NO:51, a VH CDR3 comprising the amino acid sequence of SEQ ID NO:52; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:49;
(iii) a VH region comprising a VH CDR1 comprising the amino acid sequence of SEQ ID NO:53, a VH CDR2 comprising the amino acid sequence of SEQ ID NO:47, a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49;
(iv) a VH region comprising a VH CDR1 comprising the amino acid sequence of SEQ ID NO:13, a VH CDR2 comprising the amino acid sequence of SEQ ID NO:54, a VH CDR3 comprising the amino acid sequence of SEQ ID NO:55; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:56;
(v) a VH region comprising a VH CDR1 comprising the amino acid sequence of SEQ ID NO:57, a VH CDR2 comprising the amino acid sequence of SEQ ID NO:58, a VH CDR3 comprising the amino acid sequence of SEQ ID NO:59; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:60; or
(vi) a VH region comprising a VH CDR1 comprising the amino acid sequence of SEQ ID NO:46, a VH CDR2 comprising the amino acid sequence of SEQ ID NO:61, a VH CDR3 comprising the amino acid sequence of SEQ ID NO:48; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:49.

8. The antibody or fragment thereof of any one of claims 1 to 7, wherein the antibody or fragment thereof further comprises a framework 1 (FR1), a framework 2 (FR2), a framework 3 (FR3) and/or a framework 4 (FR4) sequence as set forth in any one of SEQ ID NOs: 25, 26, 44, 45, 62, and 63.

9. The antibody or fragment thereof of any one of claims 1 to 7, wherein the antibody or fragment thereof further comprises human framework sequences.

10. The antibody or fragment thereof of any one of claims 1 to 8, wherein the antibody or fragment thereof comprises:
(i) a VH comprising the amino acid sequence of SEQ ID NO:25 and a VL comprising the amino acid sequence of SEQ ID NO:26;
(ii) a VH comprising the amino acid sequence of SEQ ID NO:44 and a VL comprising the amino acid sequence of SEQ ID NO:45; or
(iii) a VH comprising the amino acid sequence of SEQ ID NO:62 and a VL comprising the amino acid sequence of SEQ ID NO:63.

11. The antibody or fragment thereof of any one of claims 1-10, wherein the antibody is a monoclonal antibody.

12. The antibody or fragment thereof of any one of claims 1-11, wherein the antibody is a humanized, human or chimeric antibody.

13. The antibody or fragment thereof of any one of claims 1-12, which is a Fab, Fab′, F(ab′)₂, Fv, scFv, (scFv)₂, single chain antibody molecule, dual variable region antibody, single variable region antibody, linear antibody, V region, or a multispecific antibody formed from antibody fragments.

14. The antibody or fragment thereof of any one of claims 1-13, which is conjugated or recombinantly fused to a diagnostic agent, detectable agent or therapeutic agent.

15. The antibody or fragment thereof of claim 14, wherein the therapeutic agent is a chemotherapeutic agent, cytotoxin, or drug.

16. A binding agent that binds to essentially the same epitope as an antibody or fragment thereof of any one of claims 1-15.

17. The binding agent of claim 16, which is an antibody or fragment thereof.

18. The binding agent of claim 16, which comprises a non-antibody protein scaffold.

19. The binding agent of claim 18, wherein the non-antibody protein scaffold comprises a fibronectin scaffold, an anticalin, an adnectin, an affibody, a DARPin, a fynomer, an affitin, an affilin, an avimer, a cysteine-rich knottin peptide, or an engineered Kunitz-type inhibitor.

20. A binding agent that competes for binding to human 5T4 with an antibody or fragment thereof of any one of claims 1-15.

21. The binding agent of claim 20, wherein the binding agent is an antibody or fragment thereof.

22. One or more vectors comprising one or more polynucleotides encoding the antibody or fragment thereof of any one of claims 1-15 or a complementary polynucleotide.

23. A pharmaceutical composition that comprises the antibody or fragment thereof of any one of claims 1-15 or the binding agent of any one of claims 16-21, and a pharmaceutically acceptable carrier.

24. A method for treating a cancer or a tumor in a subject comprising administering to the subject the antibody or fragment thereof of any one of claims 1-15 or the pharmaceutical composition of claim 23.

25. A method for alleviating one or more symptoms associated with a cancer or a tumor in a subject comprising administering to the subject the antibody or fragment thereof of any one of claims 1-15 or the pharmaceutical composition of claim 23.

26. A method for decreasing tumor size in a subject with a tumor comprising administering to the subject the antibody or fragment thereof of any one of claims 1-15 or the pharmaceutical composition of claim 23.

27. A method for enhancing tumor cell removal in a subject with a tumor comprising administering to the subject the antibody or fragment thereof of any one of claims 1-15 or the pharmaceutical composition of claim 23.

28. A method for treating a 5T4-related disease, disorder or condition in a subject comprising administering to the subject the antibody or fragment thereof of any one of claims 1-15 or the pharmaceutical composition of claim 23.

29. The method of any one of claims 24-28, wherein the subject is administered one or more therapeutic agents in combination with the antibody or fragment thereof or the pharmaceutical composition.

30. The method of any one of claims 24-29, wherein the subject is a human subject.


SEQUENCE LISTING (89 sequences)

SEQ ID NO: 1 - protein, 10 aa, synthetic construct
  source 1..10: note = Antibody mAbA4 VH CDR1 (Exemplary, AbM)
GFTFSRYYIH

SEQ ID NO: 2 - protein, 17 aa, synthetic construct
  source 1..17: note = Antibody mAbA4 VH CDR2 (Exemplary, Kabat)
FISPGGSYTYYADSVKG

SEQ ID NO: 3 - protein, 10 aa, synthetic construct
  source 1..10: note = Antibody mAbA4 VH CDR3 (Exemplary, Kabat, AbM)
EYYSMGVLDY

SEQ ID NO: 4 - protein, 11 aa, synthetic construct
  source 1..11: note = Antibody mAbA4, mAbA15, mAbA17 VL CDR1 (Exemplary, Kabat, AbM)
RASQSVSSAVA

SEQ ID NO: 5 - protein, 7 aa, synthetic construct
  source 1..7: note = Antibody mAbA4, mAbA15, mAbA17 VL CDR2 (Exemplary, Kabat, AbM)
SASSLYS

SEQ ID NO: 6 - protein, 9 aa, synthetic construct
  source 1..9: note = Antibody mAbA4 VL CDR3 (Exemplary, IMGT, Kabat, AbM)
QQAYESPYT

SEQ ID NO: 7 - protein, 8 aa, synthetic construct
  source 1..8: note = Antibody mAbA4 VH CDR1 (IMGT)
GFTFSRYY

SEQ ID NO: 8 - protein, 8 aa, synthetic construct
  source 1..8: note = Antibody mAbA4 VH CDR2 (IMGT)
ISPGGSYT

SEQ ID NO: 9 - protein, 12 aa, synthetic construct
  source 1..12: note = Antibody mAbA4 VH CDR3 (IMGT)
AREYYSMGVLDY

SEQ ID NO: 10 - protein, 6 aa, synthetic construct
  source 1..6: note = Antibody mAbA4, mAbA15, mAbA17 VL CDR1 (IMGT)
QSVSSA

SEQ ID NO: 11 - skipped

SEQ ID NO: 12 - protein, 5 aa, synthetic construct
  source 1..5: note = Antibody mAbA4 VH CDR1 (Kabat)
RYYIH

SEQ ID NO: 13 - protein, 7 aa, synthetic construct
  source 1..7: note = Antibody mAbA4, mAbA17 VH CDR1 (Chothia)
GFTFSRY

SEQ ID NO: 14 - protein, 4 aa, synthetic construct
  source 1..4: note = Antibody mAbA4 VH CDR2 (Chothia)
PGGS

SEQ ID NO: 15 - protein, 8 aa, synthetic construct
  source 1..8: note = Antibody mAbA4 VH CDR3 (Chothia)
YYSMGVLD

SEQ ID NO: 16 - protein, 7 aa, synthetic construct
  source 1..7: note = Antibody mAbA4, mAbA15, mAbA17 VL CDR1 (Chothia)
SQSVSSA

SEQ ID NO: 17 - protein, 6 aa, synthetic construct
  source 1..6: note = Antibody mAbA4 VL CDR3 (Chothia)
AYESPY

SEQ ID NO: 18 - protein, 6 aa, synthetic construct
  source 1..6: note = Antibody mAbA4 VH CDR1 (Contact)
SRYYIH

SEQ ID NO: 19 - protein, 13 aa, synthetic construct
  source 1..13: note = Antibody mAbA4 VH CDR2 (Contact)
WVAFISPGGSYTY

SEQ ID NO: 20 - protein, 11 aa, synthetic construct
  source 1..11: note = Antibody mAbA4 VH CDR3 (Contact)
AREYYSMGVLD

SEQ ID NO: 21 - protein, 7 aa, synthetic construct
  source 1..7: note = Antibody mAbA4, mAbA15, mAbA17 VL CDR1 (Contact)
SSAVAWY

SEQ ID NO: 22 - protein, 10 aa, synthetic construct
  source 1..10: note = Antibody mAbA4, mAbA15, mAbA17 VL CDR2 (Contact)
LLIYSASSLY

SEQ ID NO: 23 - protein, 8 aa, synthetic construct
  source 1..8: note = Antibody mAbA4 VL CDR3 (Contact)
QQAYESPY

SEQ ID NO: 24 - protein, 10 aa, synthetic construct
  source 1..10: note = Antibody mAbA4 VH CDR2 (AbM)
FISPGGSYTY

SEQ ID NO: 25 - protein, 119 aa, synthetic construct
  source 1..119: note = Antibody mAbA4 VH Sequence
EVQLVESGGGLVQPGGSLRLSCAASGFTFSRYYIHWVRQAPGKGLEWVAFISPGGSYTYYADSVKGRFTISADTSKNTAYLQMNSLRAEDTAVYYCAREYYSMGVLDYWGQGTLVTVSS

SEQ ID NO: 26 - protein, 107 aa, synthetic construct
  source 1..107: note = Antibody mAbA4 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQAYESPYTFGQGTKVEIK

SEQ ID NO: 27 - protein, 10 aa, synthetic construct
  source 1..10: note = Antibody mAbA15 VH CDR1 (Exemplary, AbM)
GFTFSSYYIH

SEQ ID NO: 28 - protein, 17 aa, synthetic construct
  source 1..17: note = Antibody mAbA15 VH CDR2 (Exemplary, Kabat)
YITSTGSYTEYADSVKG

SEQ ID NO: 29 - protein, 12 aa, synthetic construct
  source 1..12: note = Antibody mAbA15 VH CDR3 (Exemplary, Kabat, AbM)
YDRSTRYSGLDY

SEQ ID NO: 30 - protein, 9 aa, synthetic construct
  source 1..9: note = Antibody mAbA15 VL CDR3 (Exemplary, IMGT, Kabat, AbM)
QQAYATPVT

SEQ ID NO: 31 - protein, 8 aa, synthetic construct
  source 1..8: note = Antibody mAbA15 VH CDR1 (IMGT)
GFTFSSYY

SEQ ID NO: 32 - protein, 8 aa, synthetic construct
  source 1..8: note = Antibody mAbA15 VH CDR2 (IMGT)
ITSTGSYT

SEQ ID NO: 33 - protein, 14 aa, synthetic construct
  source 1..14: note = Antibody mAbA15 VH CDR3 (IMGT)
ARYDRSTRYSGLDY

SEQ ID NO: 34 - protein, 5 aa, synthetic construct
  source 1..5: note = Antibody mAbA15 VH CDR1 (Kabat)
SYYIH

SEQ ID NO: 35 - protein, 7 aa, synthetic construct
  source 1..7: note = Antibody mAbA15 VH CDR1 (Chothia)
GFTFSSY

SEQ ID NO: 36 - protein, 4 aa, synthetic construct
  source 1..4: note = Antibody mAbA15 VH CDR2 (Chothia)
STGS

SEQ ID NO: 37 - protein, 10 aa, synthetic construct
  source 1..10: note = Antibody mAbA15 VH CDR3 (Chothia)
DRSTRYSGLD

SEQ ID NO: 38 - protein, 6 aa, synthetic construct
  source 1..6: note = Antibody mAbA15 VL CDR3 (Chothia)
AYATPV

SEQ ID NO: 39 - protein, 6 aa, synthetic construct
  source 1..6: note = Antibody mAbA15 VH CDR1 (Contact)
SSYYIH

SEQ ID NO: 40 - protein, 13 aa, synthetic construct
  source 1..13: note = Antibody mAbA15 VH CDR2 (Contact)
WVAYITSTGSYTE

SEQ ID NO: 41 - protein, 13 aa, synthetic construct
  source 1..13: note = Antibody mAbA15 VH CDR3 (Contact)
ARYDRSTRYSGLD

SEQ ID NO: 42 - protein, 8 aa, synthetic construct
  source 1..8: note = Antibody mAbA15 VL CDR3 (Contact)
QQAYATPV

SEQ ID NO: 43 - protein, 10 aa, synthetic construct
  source 1..10: note = Antibody mAbA15 VH CDR2 (AbM)
YITSTGSYTE

SEQ ID NO: 44 - protein, 121 aa, synthetic construct
  source 1..121: note = Antibody mAbA15 VH Sequence
EVQLVESGGGLVQPGGSLRLSCAASGFTFSSYYIHWVRQAPGKGLEWVAYITSTGSYTEYADSVKGRFTISADTSKNTAYLQMNSLRAEDTAVYYCARYDRSTRYSGLDYWGQGTLVTVSS

SEQ ID NO: 45 - protein, 107 aa, synthetic construct
  source 1..107: note = Antibody mAbA15 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQAYATPVTFGQGTKVEIK

SEQ ID NO: 46 - protein, 10 aa, synthetic construct
  source 1..10: note = Antibody mAbA17 VH CDR1 (Exemplary, AbM)
GFTFSRYWIH

SEQ ID NO: 47 - protein, 17 aa, synthetic construct
  source 1..17: note = Antibody mAbA17 VH CDR2 (Exemplary, Kabat)
AIFPYSGSTLYADSVKG

SEQ ID NO: 48 - protein, 13 aa, synthetic construct
  source 1..13: note = Antibody mAbA17 VH CDR3 (Exemplary, Kabat, AbM)
SPSRSILPSGLDY

SEQ ID NO: 49 - protein, 9 aa, synthetic construct
  source 1..9: note = Antibody mAbA17 VL CDR3 (Exemplary, IMGT, Kabat, AbM)
QQGDGSPYT

SEQ ID NO: 50 - protein, 8 aa, synthetic construct
  source 1..8: note = Antibody mAbA17 VH CDR1 (IMGT)
GFTFSRYW

SEQ ID NO: 51 - protein, 8 aa, synthetic construct
  source 1..8: note = Antibody mAbA17 VH CDR2 (IMGT)
IFPYSGST

SEQ ID NO: 52 - protein, 15 aa, synthetic construct
  source 1..15: note = Antibody mAbA17 VH CDR3 (IMGT)
ARSPSRSILPSGLDY

SEQ ID NO: 53 - protein, 5 aa, synthetic construct
  source 1..5: note = Antibody mAbA17 VH CDR1 (Kabat)
RYWIH

SEQ ID NO: 54 - protein, 4 aa, synthetic construct
  source 1..4: note = Antibody mAbA17 VH CDR2 (Chothia)
PYSG

SEQ ID NO: 55 - protein, 11 aa, synthetic construct
  source 1..11: note = Antibody mAbA17 VH CDR3 (Chothia)
PSRSILPSGLD

SEQ ID NO: 56 - protein, 6 aa, synthetic construct
  source 1..6: note = Antibody mAbA17 VL CDR3 (Chothia)
GDGSPY

SEQ ID NO: 57 - protein, 6 aa, synthetic construct
  source 1..6: note = Antibody mAbA17 VH CDR1 (Contact)
SRYWIH

SEQ ID NO: 58 - protein, 13 aa, synthetic construct
  source 1..13: note = Antibody mAbA17 VH CDR2 (Contact)
WVAAIFPYSGSTL

SEQ ID NO: 59 - protein, 14 aa, synthetic construct
  source 1..14: note = Antibody mAbA17 VH CDR3 (Contact)
ARSPSRSILPSGLD

SEQ ID NO: 60 - protein, 8 aa, synthetic construct
  source 1..8: note = Antibody mAbA17 VL CDR3 (Contact)
QQGDGSPY

SEQ ID NO: 61 - protein, 10 aa, synthetic construct
  source 1..10: note = Antibody mAbA17 VH CDR2 (AbM)
AIFPYSGSTL

SEQ ID NO: 62 - protein, 122 aa, synthetic construct
  source 1..122: note = Antibody mAbA17 VH Sequence
EVQLVESGGGLVQPGGSLRLSCAASGFTFSRYWIHWVRQAPGKGLEWVAAIFPYSGSTLYADSVKGRFTISADTSKNTAYLQMNSLRAEDTAVYYCARSPSRSILPSGLDYWGQGTLVTVSS

SEQ ID NO: 63 - protein, 107 aa, synthetic construct
  source 1..107: note = Antibody mAbA17 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQGDGSPYTFGQGTKVEIK

SEQ ID NO: 64 - protein, 420 aa, Homo sapiens
  source 1..420: note = full-length amino acid sequence of human 5T4
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 65 - protein, 420 aa, Macaca fascicularis
  source 1..420: note = full-length amino acid sequence of cynomolgous monkey (cyno) 5T4
MPGGCSRGPAAGDGRLRLARLALVLLGWVSSSSSTSSASSSSSSAPFLASAASAQPPLPDQCPALCECSEAARTVKCVNRNLTEVPTDLPLYVRNLFLTGNQLAVLPAGAFARRPPLAELAALNLSGSRLDEVRGGAFEHLPSLRQLDLSHNPLAYLSPFAFSGSNASISAPSPLVELILNHIVPPDDKRQNRSFEGMVAAALVAGRALQGLHLLELASNHFLYLPRDVLAQLPSLRYLDLSNNSLVSLTYVSFRNLTHLESLHLEDNALKVLHNGTLAELQGLPHVRVFLDNNPWVCDCHMADMVTWLKQTGVVQGKDRLTCAFPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 66 - protein, 426 aa, Mus musculus
  source 1..426: note = full-length amino acid sequence of mouse 5T4
MPGAGSRGPSAGDGRLRLARLALVLLGWVSASAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTSYVFLGIVLALIGAIFLLVLYLNRKGIKKWMHNIRDACRDHMEGYHYRYEINADPRLTNLSSNSDV

SEQ ID NO: 67 - protein, 222 aa, synthetic construct
  source 1..222: note = exemplary Fc region sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 222 aa, synthetic construct
  source 1..222: note = exemplary variant Fc region (silent Fc)
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 330 aa, synthetic construct [sequence omitted]

SEQ ID NO: 70 - protein, 330 aa, synthetic construct
  source 1..330: note = exemplary IgG heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 107 aa, synthetic construct
  source 1..107: note = exemplary light chain (e.g., for pairing with an IgG heavy chain)
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 226 aa, synthetic construct
  source 1..226: note = phage display heavy chain
  VARIANT 31..35: note = X is any amino acid
  VARIANT 50..66: note = X is any amino acid
  VARIANT 99..112: note = X is any amino acid
EVQLVESGGGLVQPGGSLRLSCAASGFTFSXXXXXWVRQAPGKGLEWVAXXXXXXXXXXXXXXXXXRFTISADTSKNTAYLQMNSLRAEDTAVYYCARXXXXXXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 73 - protein, 214 aa, synthetic construct
  source 1..214: note = phage display light chain
  VARIANT 89..97: note = X is any amino acid
DIQMTQSPSSLSASVGDRVTITCRASQSVSSAVAWYQQKPGKAPKLLIYSASSLYSGVPSRFSGSRSGTDFTLTISSLQPEDFATYYCXXXXXXXXXFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 330 aa, synthetic construct [sequence omitted]

SEQ ID NO: 75 - protein, 107 aa, synthetic construct; identical to SEQ ID NO: 71
  source 1..107: note = full length IgGs, the light chain constant region

SEQ ID NO: 76 - protein, 43 aa, Homo sapiens
  source 1..43: note = Epitope 1 of human 5T4 (H1)
SSPTSSASSFSSSAPFLASAVSAQPPLPDQCPALCECSEAART

SEQ ID NO: 77 - protein, 60 aa, Homo sapiens
  source 1..60: note = Epitope 2 of human 5T4 (H2)
VKCVNRNLTEVPTDLPAYVRNLFLTGNQLAVLPAGAFARRPPLAELAALNLSGSRLDEVR

SEQ ID NO: 78 - protein, 30 aa, Homo sapiens
  source 1..30: note = Epitope 3 of human 5T4 (H3)
AGAFEHLPSLRQLDLSHNPLADLSPFAFSG

SEQ ID NO: 79 - protein, 52 aa, Homo sapiens
  source 1..52: note = Epitope 4 of human 5T4 (H4)
SNASVSAPSPLVELILNHIVPPEDERQNRSFEGMVVAALLAGRALQGLRRLE

SEQ ID NO: 80 - protein, 47 aa, Homo sapiens
  source 1..47: note = Epitope 5 of human 5T4 (H5)
LASNHFLYLPRDVLAQLPSLRHLDLSNNSLVSLTYVSFRNLTHLESL

SEQ ID NO: 81 - protein, 28 aa, Homo sapiens
  source 1..28: note = Epitope 6 of human 5T4 (H6)
HLEDNALKVLHNGTLAELQGLPHIRVFL

SEQ ID NO: 82 - protein, 64 aa, Homo sapiens
  source 1..64: note = Epitope 7 of human 5T4 (H7)
DNNPWVCDCHMADMVTWLKETEVVQGKDRLTCAYPEKMRNRVLLELNSADLDCDPILPPSLQTS

SEQ ID NO: 83 - protein, 330 aa, synthetic construct
  source 1..330: note = Human 5T4 Epitope 1
SSPTSSASSFSSSAPFLASAVSAQPPLPDQCPALCECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 84 - protein, 330 aa, synthetic construct
  source 1..330: note = Human 5T4 Epitope 2
SAPSSSVPSSSTSPAAFLASGSAQPPPAERCPAACECSEAARTVKCVNRNLTEVPTDLPAYVRNLFLTGNQLAVLPAGAFARRPPLAELAALNLSGSRLDEVRAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 85 - protein, 330 aa, synthetic construct
  source 1..330: note = Human 5T4 Epitope 3
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPSLRQLDLSHNPLADLSPFAFSGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 86 - protein, 324 aa, synthetic construct
  source 1..324: note = Human 5T4 Epitope 4
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLVELILNHIVPPEDERQNRSFEGMVVAALLAGRALQGLRRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 87 - protein, 330 aa, synthetic construct
  source 1..330: note = Human 5T4 Epitope 5
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLYLPRDVLAQLPSLRHLDLSNNSLVSLTYVSFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 88 - protein, 330 aa, synthetic construct
  source 1..330: note = Human 5T4 Epitope 6
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNGTLAELQGLPHIRVFLDNNPWVCDCYMADMVAWLKETEVVPDKARLTCAFPEKMRNRGLLDLNSSDLDCDAVLPQSLQTS

SEQ ID NO: 89 - protein, 330 aa, synthetic construct
  source 1..330: note = Human 5T4 Epitope 7
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCHMADMVTWLKETEVVQGKDRLTCAYPEKMRNRVLLELNSADLDCDPILPPSLQTS